(19) United States
(12) Patent Application Publication
(10) Pub. No.: US 2009/0035394 A1
(43) Pub. Date: Feb. 5, 2009

(54) USE OF DNA-PK INHIBITION TO SENSITISE ATM DEFICIENT CANCERS TO DNA-DAMAGING CANCER THERAPIES

(76) Inventors: Graeme Cameron Murray Smith, Cambridge (GB); Andrew James Slade, Cambridge (GB); Caroline Jane Richardson, Cambridge (GB); Barbara Wanda Durkacz, Newcastle upon Tyne (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/915,282

(22) PCT Filed: May 26, 2006
(86) PCT No.: PCT/GB2006/001946

§ 371 Date: May 7, 2008

Related U.S. Application Data
(60) Provisional application No. 60/685,582, filed on May 26, 2005.

(30) Foreign Application Priority Data
May 26, 2005 (GB) 0510770.1

(51) Int. Cl. A61K 33/24; A61K 31/5377; A61K 31/7048; A61P 35/00; A61K 31/704

(52) U.S. Cl. 424/649; 514/233.5; 514/27; 514/34

(57) ABSTRACT

This invention relates to the finding that inhibition of the catalytic subunit of DNA protein kinase (DNA-PKcs) increases the sensitivity of cancer cells that display an ATM deficient phenotype to DNA damaging therapies, such as irradiation or chemotherapy. Methods of treating cancers displaying an ATM deficient phenotype and methods of determining the susceptibility of a patient to such methods are provided.

This invention relates to the treatment of cancer conditions, in particular, cancer conditions that display an ATM deficient phenotype.

Human DNA is constantly under attack from reactive oxygen intermediates principally from by-products of oxidative metabolism. Reactive oxygen species are capable of producing DNA single-strand breaks and, where two of these are generated in close proximity, DNA double strand breaks (DSBs). In addition, single- and double-strand breaks can be induced when a DNA replication fork encounters a damaged template, and are generated by exogenous agents such as ionising radiation (IR) and certain anti-cancer drugs (e.g. bleomycin, etoposide, doxorubicin or irinotecan). DSBs also occur as intermediates in site-specific V (D) J recombination, a process that is critical for the generation of a functional vertebrate immune system. If DNA DSBs are left unrepaired or are repaired inaccurately, mutations and/or chromosomal aberrations are induced, which in turn may lead to cell death. To combat the serious threats posed by DNA DSBs, eukaryotic cells have evolved several mechanisms to mediate their repair. Critical to the process of DNA repair is the slowing down of cellular proliferation to allow time for the cell to repair the damage. A key protein in the detection of DNA DSBs and in the signalling of this information to the cell cycle machinery is the kinase ATM (ataxia telangiectasia mutated) (Durocher and Jackson (2001) Curr Opin Cell Biol. 13: 225-31, Abraham (2001) Genes Dev. 15; 2177-96).

The ATM protein is a 350 kDa polypeptide that is a member of the phosphatidylinositol (PI) 3-kinase family of proteins by virtue of a putative kinase domain in its carboxyl-terminal region (Savitsky et al (1995) Science, 268: 1749-53). Classical PI 3-kinases, such as PI 3-kinase itself, are involved in signal transduction and phosphorylate inositol lipids that act as intracellular second messengers (reviewed in Toker and Cantley (1997), Nature, 387: 673-6).

However, ATM bears most sequence similarity with a subset of the PI 3-kinase family that phosphorylate proteins and which, like ATM, are involved in cell cycle control and/or in the detection and signalling of DNA damage. This subset of kinases are known as the phosphatidylinositol-3 kinase related kinases (PIKKs) (Keith and Schreiber (1995), Science, 270; 50-1, Zakian (1995) Cell, 82; 685-7). Notably there is no evidence to date that any members of the PIKK family are able to phosphorylate lipids. However, all members of the PIKK family have been shown to possess serine/threonine kinase activity. ATM phosphorylates key proteins involved in a variety of cell-cycle checkpoint signalling pathways that are initiated in response to DNA DSBs production.

ATM is the product of the gene mutated in ataxia-telangiectasia (A-T) (Savitsky et al (1995)). A-T is a human autosomal recessive disorder present at an incidence of around 1 in 100,000 in the population. A-T is characterised by a number of debilitating symptoms, including progressive cerebellar degeneration, occulocutaneous telangiectasia, growth retardation, immune deficiencies, cancer predisposition and certain characteristics of premature ageing (Lavin and Shiloh (1997), Annu. Rev. Immunol., 15: 177-202; Shiloh (2001), Curr. Opin. Genet. Dev. 11: 71-7). At the cellular level, A-T is characterised by a high degree of chromosomal instability, radio-resistant DNA synthesis, and hypersensitivity to ionizing radiation (1R) and radiomimetic drugs. In addition, A-T cells are defective in the radiation induced G1-S, S, and G2-M cell cycle checkpoints that are thought to arrest the cell cycle in response to DNA damage in order to allow repair of the genome prior to DNA replication or mitosis (Lavin and Shiloh, 1997). This may in part reflect the fact that A-T cells exhibit deficient or severely delayed induction of p53 in response to IR. Indeed, p53-mediated downstream events are also defective in A-T cells following IR exposure. ATM therefore acts upstream of p53 in an IR-induced DNA damage signalling pathway.

A-T cells have also been shown to accumulate DNA double-strand breaks (DSBs) after ionising radiation, suggesting a defect in DSB repair. ATM function in response to ionising radiation induced DNA damage has been shown to be tissue specific. For example, while fibroblasts derived from ATM null mice are radiosensitive, ATM null neurons are radioresistant through a lack of IR induced apoptosis (Herzog et al. (1998) Science, 280: 1089-91).

The present inventors have discovered that the sensitivity of cancer cells which display an ATM deficient phenotype to the effect of certain DNA damaging anti-cancer therapies can be increased by inhibition of DNA protein kinase (DNA-PK) and, in particular, the catalytic subunit thereof (DNA-PKcs). This has important implications in the treatment of cancer conditions.

### SUMMARY OF THE INVENTION

One aspect of the invention provides the use of a combination of a DNA-PKcs inhibitor and a DNA damaging cancer therapy in the manufacture of a medicament for use in the treatment of cancer in an individual,
wherein said cancer has an ATM deficient phenotype.

Another aspect of the invention provides a method of treatment of cancer in an individual comprising;
administering a combination of a DNA-PKcs inhibitor and a DNA damaging cancer therapy to said individual,
wherein said cancer has an ATM deficient phenotype.

Another aspect of the invention provides the use of a DNA-PKcs inhibitor in the manufacture of a medicament for use in increasing the sensitivity of cancer cells in an individual to a DNA damaging cancer therapy,
wherein said cancer cells have an ATM deficient phenotype.

Another aspect of the invention provides a method of increasing the sensitivity of a cancer cell in an individual to a DNA damaging cancer therapy comprising;
administering a DNA-PKcs inhibitor to said individual, wherein said cancer cell has an ATM deficient phenotype.

Another aspect of the invention provides a method of determining the susceptibility of a cancer condition in an individual to a cancer therapy, said method comprising;
identifying a cancer cell obtained from the individual as having an ATM deficient phenotype,
wherein said cancer therapy comprises a combination of a DNA-PKcs inhibitor and a DNA damaging cancer therapy; and,
wherein the identification of the cancer cell obtained from the individual as a cancer cell having an ATM deficient phenotype is indicative of the cancer being susceptible to said cancer therapy.

The DNA damaging cancer therapy may be irradiation or one or more DNA damaging chemotherapeutic agents that either directly or indirectly causes DNA double stranded breaks requiring repair by the DNA-PK DNA repair pathway (also known as the non-homologous end joining pathway or NHEJ: Smith G C and Jackson S P (1999) Genes Dev 13, 916-934).

### BRIEF DESCRIPTION OF THE DRAWINGS

FIG. 1 shows a graphical representation of the increased sensitisation to the effect of etoposide (a semi-synthetic podophyllotoxin derivative DNA topoisomerase II inhibitor) of ATM-null cells by the specific ATP-competitive DNA-PKcs inhibitor KU-0060648.

FIG. 2 shows a graphical representation of the increased sensitivity to the effect of doxorubicin (an anthracycline antibiotic DNA topoisomerase II inhibitor) of ATM-null cells treated with the specific ATP-competitive DNA-PKcs inhibitor KU-0060648.

FIG. 3 shows a graphical representation of the increased sensitivity to the effect of ionising radiation of ATM-null cells treated with the specific ATP-competitive DNA-PKcs inhibitor KU-0060648.

### DETAILED DESCRIPTION OF THE INVENTION

DNA-PK is a Ser/Thr kinase which is a heterotrimer of DNA-PKcs (P78527 GI:38258929) and the Ku p70/p86 dimer (G22P1/G22P2; CAG30378.1, GI:47678515, P13010). DNA-PK is a key component of the DNA non-homologous end joining (NHEJ) pathway and is required for double-strand break repair and V(D)J recombination.

The present invention, in various aspects, relates to the use of DNA-PKcs inhibitors to increase the sensitivity of cancer cells which display an ATM deficient phenotype to the effect of DNA damaging anti-cancer therapies, such as irradiation or chemotherapy which directly or indirectly causes DNA double stranded breaks.

An increase in sensitivity of a cancer cell to a chemotherapeutic agent (also be referred to as ‘sensitisation’ or ‘hypersensitisation’) is defined as an increase in the therapeutic index of the chemotherapeutic agent against the cancer cell.

The invention encompasses a DNA-PKcs inhibitor as described herein for use in combination with a DNA damaging chemotherapeutic agent in the treatment of cancer in an individual, wherein said cancer has an ATM deficient phenotype. The invention also encompasses a DNA damaging chemotherapeutic agent as described herein for use in combination with a DNA-PKcs inhibitor in the treatment of cancer in an individual, wherein said cancer has an ATM deficient phenotype.

A DNA-PKcs inhibitor is a biological or preferably a chemical entity that specifically interacts with the catalytic subunit of DNA-PK (DNA-PKcs), for example in an allosteric manner or, more preferably, in an ATP competitive manner, and reduces or abolishes its kinase activity, thereby inhibiting the DNA repair function of DNA-PK.

A DNA-PKcs inhibitor that specifically interacts with DNA-PKcs preferably shows no binding or substantially no binding to protein kinases of the PIKK family. A suitable DNA-PKcs inhibitor may show at least 100 fold, at least 1000 fold or 10000 fold greater binding to DNA-PKcs than to a kinase of the PIKK family. For example, a DNA-PKcs inhibitor may display an IC50 of less than 50 nM for DNA-PKcs and an IC50 of greater than 5 μM or greater for members of the PIKK family.

In some embodiments, a specific DNA-PKcs inhibitor may some cross-reaction with PI3 kinase (i.e. the DNA-PKcs inhibitor may bind to PI3 kinase as well as DNA-PKcs).

Preferred DNA-PKcs inhibitors for use in accordance with the present methods reversibly interact with DNA-PKcs and do not form covalent bonds. Reversible DNA-PKcs inhibitors are known in the art and are described in more detail below.

Cancers with an ATM deficient phenotype include cancers in which the ATM-mediated homologous repair activity of some or all of the cancer cells is reduced or ablated compared to non-tumour tissue through either the absence (ATM-null), reduction in amount or dysfunction of the ATM protein.

A individual suitable for treatment as described herein may include a eukaryote, an animal, a vertebrate animal, a mammal, a rodent (e.g. a guinea pig, a hamster, a rat, a mouse), a murine (e.g. a mouse), a canine (e.g. a dog), a feline (e.g. a cat), an equine (e.g. a horse), a primate, such as a simian (e.g. a monkey or ape), a monkey (e.g. marmoset, baboon), an ape (e.g. gorilla, chimpanzee, orang-utan, gibbon), or a human.

Cancer cells in general are characterised by abnormal proliferation relative to normal cells and typically form clusters or tumours in an individual having a cancer condition. Cancers with an ATM deficient phenotype include cancers which comprise one or more cancer cells which have a reduced or abrogated ability to repair DNA DSBs through the ATM-dependent DNA damage checkpoint pathway, relative to normal cells i.e. the activity of the ATM-dependent DNA damage checkpoint pathway is reduced or abolished in the one or more cancer cells. In some preferred embodiments, the activity of the ATM-dependent DNA damage checkpoint pathway is reduced by 40% or more, 50% or more, 60% or more, 70% or more, 80% or more, or 90% or more, in cells with an ATM deficient phenotype, relative to normal cells.

Cancers with an ATM deficient phenotype include cancers which have an ATM null phenotype. Cancers with an ATM null phenotype comprise one or more cancer cells which are not able to repair DNA DSBs through the ATM-dependent DNA damage checkpoint pathway i.e. the activity of the ATM-dependent DNA damage checkpoint pathway is abolished in the one or more cancer cells.

In preferred embodiments, the ATM deficient phenotype is characteristic of cancer cells from the individual and non-cancer cells from the individual do not have the ATM deficient phenotype i.e. healthy cells from the individual have normal ability to repair DNA DSBs through the ATM-dependent DNA damage checkpoint pathway and the activity of the ATM-dependent DNA damage checkpoint pathway is not reduced or impaired. For example, in some embodiments, an individual with the cancer condition is not an individual suffering from Ataxia telangiectasia (A-T) or other condition caused by a general dysfunction in the homologous repair pathway, such as Nijmegen Breakage Syndrome (Weemaes C M et al. (1981) Acta Paediatr. Scand. 70, 557-564).

The ATM dependent DNA damage checkpoint response pathway is described in more detail in Khanna and Jackson (2001) Nat. Genet. 27, 247-254 and Lobrich M and Jeggo P A (2005) Radiother. Oncol. 76, 112-118.

The nucleic acid and protein sequences of ATM are available from the GenBank database, under the following accession numbers: Human ATM (Nucleic acid coding sequence (CDS): U82828.1 GI: 2304970, protein sequence: AAB65827.1 GI: 2304971.

Cancers with an ATM deficient phenotype include cancers deficient in ATM itself i.e. cancers in which the expression and/or activity of the ATM protein is reduced or abolished, for example by means of mutation, polymorphism or hypermethylation in the encoding nucleic acid, or by means of mutation, polymorphism or hypermethylation in a regulatory region or a gene encoding a regulatory factor.

ATM deficiency may be due to mutations in the coding region of the ATM gene that prevent the translation of full-length active protein i.e. truncating mutations, mutations in the coding region of the ATM gene that allow the translation of full-length but inactive or impaired function protein i.e. missense mutations, mutations in the regulatory elements of the ATM gene that prevent transcription or epigenetic changes that prevent transcription, for example methylation in the regulatory elements of the ATM gene. Examples of mutations in the ATM gene which are known to lead to ATM deficiency are shown in Table 9. Other known mutations in the ATM gene may be found in the on-line Ataxia-Telangiectasia Mutation Database, Concannon P., Benaroya Research Institute, Seattle, Wash. 98101.

In some embodiments, an ATM deficient cancer cell is a cell which has less than 50%, less than 40%, less than 30%, less than 20% or less than 10% of the normal population level of active ATM protein (Thompson et al (2005) J. Natl. Cancer Inst. 97, 813-822). An ATM null cancer cell is a cell which contains no active ATM protein or substantially no active ATM protein.

In some embodiments, cancers with an ATM deficient phenotype include cancers derived from a cell lineage that has low ATM expression or activity. For example, most follicular centre-cell lymphomas (FCCL) and diffuse large B-cell lymphomas (DLBCL), which rarely show inactivation of the ATM gene, are negative or stain weakly for ATM protein. These tumours are derived from cells where ATM expression is down-regulated to undetectable or very low levels during normal cell differentiation to accommodate developmentally programmed DNA double stranded breaks i.e. the patterns of ATM expression seen within B-cell tumours reflects the individual stages of B-cell differentiation from which tumours are derived (Starczynski J, Simmons W, et al (2003) Am J Pathol 163, 423-32).

Cells deficient in ATM include cells which are heterozygous or homozygous for expression or activity-reducing mutations or polymorphisms in the nucleic acid encoding the ATM gene or its regulatory elements.

An ATM deficient phenotype may be displayed by any type of solid cancer for example, sarcomas, skin cancer, bladder cancer, breast cancer, uterine cancer, ovarian cancer, prostate cancer, lung cancer, colorectal cancer, cervical cancer, liver cancer, head and neck cancer, oesophageal cancer, pancreatic cancer, renal cancer, stomach cancer and cerebral cancer. ATM deficient phenotypes are particularly common in lymphoid cancers, such as leukaemia or lymphoma, in particular follicular centre-cell lymphoma (FCCL), chronic lymphocytic leukaemia (CLL), mantle cell lymphoma (MCL) and diffuse large B-cell lymphoma (DLBCL).

In the methods described herein, a cancer condition in an individual may have been previously identified as a cancer having an ATM deficient phenotype or a method may comprise the step of identifying a cancer condition in an individual as having an ATM deficient phenotype. Cancer conditions identified as having an ATM deficient phenotype are suitable for treatment as described herein. A cancer may be identified as having an ATM deficient phenotype by a range of approaches which are well known the art.

In some embodiments, a cancer may be identified as having an ATM deficient phenotype by determining the presence in cancer cells from the individual of one or more variations, for example, polymorphisms, mutations or regions of hypermethylation, in a nucleic acid encoding a polypeptide which is a component of the ATM dependent DNA damage checkpoint response pathway, such as ATM. For example, the presence of an ATM gene mutation shown in Table 9 may be determined.

Sequence variations, such as mutations and polymorphisms may include a deletion, insertion or substitution of one or more nucleotides, relative to the wild-type nucleotide sequence. In some embodiments, the variation may be a gene amplification or an increase or decrease in methylation. The one or more variations may be in a coding or non-coding region of the nucleic acid sequence and may reduce or abolish the expression or activity of the polypeptide. In other words, the variant nucleic acid may encode a variant polypeptide which has reduced or abolished activity or may encode a wild-type polypeptide which has little or no expression within the cell, for example through the altered activity of a regulatory element. A variant nucleic acid may have one, two, three, four or more mutations or polymorphisms relative to the wild-type sequence.

The presence of one or more variations in a nucleic acid which encodes a component of the ATM dependent DNA damage checkpoint response pathway, such as ATM, may be determined by detecting, in one or more cells of a test sample, the presence of the variant nucleic acid sequence or by detecting the presence of the variant polypeptide which is encoded by the nucleic acid sequence.

A cancer may also be identified as having an ATM deficient phenotype by determining the level in cancer cells from the individual, of nucleic acid, for example mRNA, encoding a polypeptide which is a component of the ATM dependent DNA damage checkpoint response pathway, such as ATM. Reduced levels or absence of the nucleic acid relative to controls may be indicative of an ATM deficient phenotype.

Various methods are available for determining the presence or absence or level in a sample of cells obtained from an individual of a particular nucleic acid sequence, for example a nucleic acid encoding an ATM dependent DNA damage checkpoint response pathway component or a variant thereof which has a mutation or polymorphism, including for example, single-strand conformation polymorphism (SSCP) (Castellvi-Bel et al Human Mutation 14 2 156-162). Many other suitable methods are known in the art and are described, for example, in Molecular Cloning: a Laboratory Manual: 3rd edition, Sambrook & Russell (2001) Cold Spring Harbor Laboratory Press NY and Current Protocols in Molecular Biology, Ausubel et al. eds. John Wiley & Sons (1992).

Nucleic acid or an amplified region thereof, may be sequenced to identify or determine the presence of polymorphism or mutation therein. A polymorphism or mutation may be identified by comparing the sequence obtained with the known sequence of the component, for example as set out in sequence databases. In particular, the presence of one or more polymorphisms or mutations that cause abrogation or loss of function of the polypeptide component, and thus the ATM dependent DNA DSB repair pathway as a whole, may be determined. Sequencing may be performed using any one of a range of standard techniques. Sequencing of an amplified product may, for example, involve precipitation with isopropanol, resuspension and sequencing using a TaqFS+Dye terminator sequencing kit. Extension products may be electrophoresed on an ABI 377 DNA sequencer and data analysed using Sequence Navigator software.

Having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

More preferably, cancer may be identified at the protein level as having an ATM deficient phenotype by determining the presence and preferably the amount of a polypeptide which is a component of the ATM dependent DNA damage checkpoint response pathway, such as ATM, in tumour cells from an individual. Low levels or absence of the polypeptide relative to controls may be indicative of an ATM deficient phenotype. Many suitable methods may be employed, including, for example, Western blot analysis, immunohistochemistry (Angèle S et al (2000) Clin. Cancer Res. 6, 3536-3544) or by immunoassay (Butch A W et al (2004) Clinical Chemistry 50, 2303-2308).

A cancer may also be identified at the protein level as having an ATM deficient phenotype by determining the presence and/or amount of a polypeptide which is a component of the ATM dependent DNA damage checkpoint response pathway, such as ATM, in cancer cells from the individual. Many suitable methods may be employed, including, for example, western blotting or immunohistology. The absence of the polypeptide or reduced amounts relative to controls, for example less than 50%, may be indicative of an ATM deficient phenotype.

Mutations and polymorphisms associated with cancer may also be detected by detecting the presence of a variant polypeptide (i.e. a mutant or allelic variant with reduced activity).

A method of identifying a cancer cell in a sample from an individual as ATM deficient may comprise contacting a sample with a specific binding member, for example an antibody, directed against ATM, and determining binding of the specific binding member to the sample. Binding of the specific binding member to the sample may be indicative of the presence of ATM in a cell within the sample. The amount of binding of the specific binding member to the sample may be indicative of the level or amount of ATM in a cell within the sample.

The reactivity of a binding member such as an antibody on normal and test samples may be determined by any appropriate means. The mode of determining binding is not a feature of the present invention and those skilled in the art are able to choose a suitable mode according to their preference and general knowledge.

In some embodiments, a cancer may be identified as having an ATM deficient phenotype by determining the activity of the ATM dependent DNA damage checkpoint response pathway in one or more cancer cells from a sample obtained from the individual. Activity may be determined relative to normal (i.e. non-cancer) cells, preferably from the same tissue. Reduced activity in the one or more cancer cells, for example less than 50%, less than 40%, less than 30%, less than 20% or less than 10%, relative to the activity of the pathway in normal (i.e. non-cancer) cells (i.e. homozygous for full length active ATM), is indicative that the cancer has an ATM deficient phenotype. Zero activity in the one or more cancer cells relative to the activity of the pathway in normal (i.e. non-cancer) cells, is indicative that the cancer has an ATM null phenotype.

The activity of the ATM dependent DNA damage checkpoint response pathway may be determined by measuring the formation of foci containing Rad51 in the nucleus in response to DNA damaging agents. Cells deficient in the ATM dependent DNA DSB repair pathway lack the ability to produce such foci. The presence of Rad51 foci may be determined using standard immunofluorescent techniques. Other methods for determining the presence of an ATM deficient phenotype may include sensitivity to IR, chemotherapeutics such as inter-strand cross linking reagents, DSB inducing agents (topoisomerase I & II inhibitors) as well as the use of western blot analysis, immunohistology, chromosomal abnormalities, enzymatic or DNA binding assays and plasmid-based assays.

Suitable samples obtained from an individual include a tissue sample comprising one or more cells, for example a biopsy from a cancerous tissue as described above, or a non-cancerous tissue, for example for use as a control.

DNA-PKcs inhibitors suitable for use in the present methods include any compound or entity, such as a small organic molecule, peptide or nucleic acid, which induces a DNA-PKcs deficient phenotype in a cell i.e. it inhibits, reduces or abolishes the activity of DNA-PKcs. DNA-PKcs inhibitors may be identified using standard techniques for example, by determining the DNA-PKcs mediated phosphorylation of a substrate using immunochemical techniques, as described herein. Suitable DNA-PKcs inhibitors include small molecule ATP-competitive kinase inhibitors which inhibit DNA-PKcs in an ATP-competitive manner.

DNA-PKcs inhibitors have been described previously. Some of these compounds inhibit DNA-PKcs irreversibly, for example by forming a covalent bond with the DNA-PKcs molecule. Wortmannin is known to irreversibly inactivate members of the phosphoinositol-3-kinase family (Arcaro and Wymann (1993) Biochem J 296, 297-301) and its interaction with DNA-PKcs is believed to underlie its activity as a radiopotentiator (Hashimoto et al (2003) J. Radiat. Res. 44, 151-159. Vanillin and structurally related benzaldehyde derivatives such as 4,5-dimethoxy-2-nitrobenzaldehyde (DMNB: Calbiochem) also inhibit DNA-PKcs irreversibly and are believed to have a similar mechanism of action to wortmannin (Durant and Karran (2003) Nucleic Acids Research 31, 5501-5512).

Preferred DNA-PKcs inhibitors inhibit DNA-PKcs reversibly. Suitable reversible DNA-PKcs inhibitors for use as described herein include: arylmorpholine 2-Hydroxy-4-morpholin-4-yl-benzaldehyde (IC60211: Calbiochem), and derivatives 1-(2-Hydroxy-4-morpholin-4-yl-phenyl)ethanone (IC86621: Calbiochem) and 1-(2-Hydroxy-4-morpholin-4-yl-phenyl)-phenyl-methanone (AMA37: Calbiochem; Kashishian et al (2003) Molecular Cancer Therapeutics 2, 1257-1264).

Other suitable DNA-PKcs inhibitors include chromenones such as 8-Dibenzothiophen-4-yl-2-morpholin-4-yl-chromen-4-one (NU7441) (Leahy et al (2004) Bioorg. Med. Chem. Lett. 14, 6083-6087) and benzochromenone 2-(Morpholin-4-yl)-benzo[h]chromen-4-one (NU7026: Calbiochem) (Willmore et al (2004) Blood 103, 4659-4665).

Other suitable DNA-PKcs inhibitors include compounds having the formula (I):

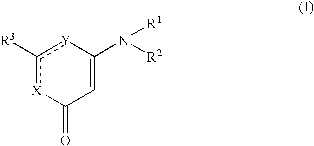

wherein:
R¹ and R² are independently hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
X and Y are selected from CR⁴ and O, O and CR¹⁴ and NR″⁴ and N, where the unsaturation is in the appropriate place in the ring, and where one of R³ and R⁴ or R¹⁴ is an optionally substituted C₃₋₂₀ heteroaryl or C₅₋₂₀ aryl group, and the other of R³ and R⁴ or R¹⁴ is H, or R³ and R⁴ or R¹⁴ together are -A-B-, which collectively represent a fused optionally substituted aromatic ring;
except that when X and Y are CR⁴ and O, R³ and R⁴ together form a fused benzene ring, and R¹ and R² together with the N to which they are attached form a morpholino group, then the fused benzene does not bear as a sole substituent a phenyl substituent at the 8-position,
or be isomers, salts, solvates, chemically protected forms, and prodrugs thereof.

Thus, the three different possibilities for X and Y results in compounds of formulae Ia, Ib and Ic:

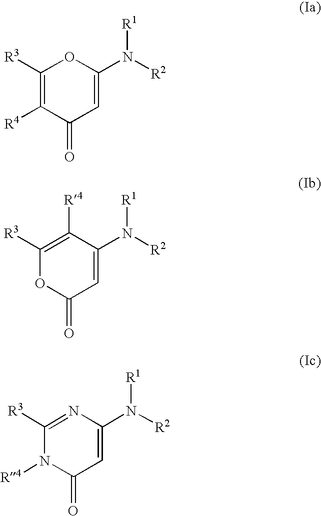

DNA-PKcs inhibitors include compounds of formulae Ia or Ib, where one R³ and R⁴ (or R′⁴) is a C₃₋₂₀ heteroaryl or C₅₋₂₀ aryl group, and the other of R³ and R⁴ (or R′⁴) is H.

DNA-PKcs inhibitors include compounds of formulae Ia and Ic, where R³ and R⁴ or R″⁴ together are -A-B-, which collectively represent a fused optionally substituted aromatic ring, with the proviso given above.

DNA-PKcs inhibitors include compounds of formula (II):

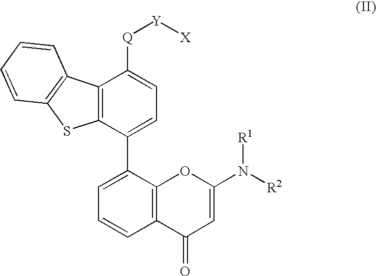

wherein:
R¹ and R² are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;

### Q is —NH—C(═O)— or —O—;

Y is an optionally substituted C₁₋₅ alkylene group;
X is selected from SR³ or NR⁴R⁵, wherein,
R³, or R⁴ and R⁵ are independently selected from hydrogen, optionally substituted C₁₋₇ alkyl, C₅₋₂₀ aryl, or C₃₋₂₀ heterocyclyl groups, or R⁴ and R⁵ may together form, along with the nitrogen atom to which they are attached, an optionally substituted heterocyclic ring having from 4 to 8 ring atoms; if Q is —O—, X is additionally selected from —C(═O)—NR⁶R⁷, wherein R⁶ and R⁷ are independently selected from hydrogen, optionally substituted C₁₋₇ alkyl, CO₅₋₂₀ aryl, or C₃₋₂₀ heterocyclyl groups, or R⁶ and R⁷ may together form, along with the nitrogen atom to which they are attached, an optionally substituted heterocyclic ring having from 4 to 8 ring atoms; and
if Q is —NH—C(═O)—, —Y—X may additionally selected from C₁₋₇ alkyl.

Preferred DNA-PKcs inhibitors of formula (II) include 8-aryl-2-morpholin-4-yl-1-benzopyran-4-one and 2-(4-Ethyl-piperazin-1-yl)-N-[4-(2-morpholin-4-yl-4-oxo-4H-1-benzopyran-8-yl)-dibenzothiophen-1-yl]-acetamide (KU-0060648).

DNA-PKcs inhibitors include compounds of formula (III):

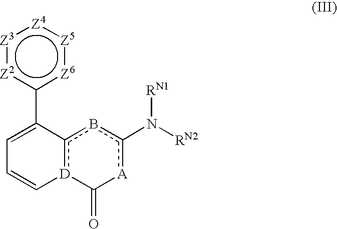

wherein:
A, B and D are respectively selected from the group consisting of:

### (i) CH, NH, C;

### (ii) CH, N,N; and

(iii) CH, O, C;
the dotted lines represent two double bonds in the appropriate locations;
R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
Z², Z³, Z⁴, Z⁵ and Z⁶, together with the carbon atom to which they are bound, form an aromatic ring;
Z² is selected from the group consisting of CR², N, NH, S, and O; Z³ is CR³; Z⁴ is selected from the group consisting of CR⁴, N, NH, S, and O; Z⁵ is a direct bond, or is selected from the group consisting of O, N, NH, S, and CH; Z⁶ is selected from the group consisting of O, N, NH, S, and CH;

### R² is H;

R³ is selected from halo or optionally substituted CO₅₋₂₀ aryl;
R⁴ is selected from the group consisting of H, OH, NO₂, NH₂ and Q-Y—X, where

### Q is —NH—C(═O)— or —O—;

Y is an optionally substituted C₁₋₅ alkylene group;
X is selected from SR^{S1} or NR^{N3}R^{N4}, wherein,
R^{S1}, or R^{N3} and R^{N4} are independently selected from hydrogen, optionally substituted C₁₋₇ alkyl, C₅₋₂₀ aryl, or C₃₋₂₀ heterocyclyl groups, or R^{N3} and R^{N4} may together form, along with the nitrogen atom to which they are attached, an optionally substituted heterocyclic ring having from 4 to 8 ring atoms;
if Q is —O—, X may additionally be selected from —C(═O)—NR^{N5}R^{N6}, wherein R^{N5} and R^{N6} are independently selected from hydrogen, optionally substituted C₁₋₇ alkyl, C₅₋₂₀ aryl, or C₃₋₂₀ heterocyclyl groups, or R^{N5} and R^{N6} may together form, along with the nitrogen atom to which they are attached, an optionally substituted heterocyclic ring having from 4 to 8 ring atoms and
if Q is —NH—C(═O)—, —Y—X may be additionally selected from C₁₋₇ alkyl.
Z², Z³, Z⁴, Z⁵ and Z⁶ are selected such that the group they form including the carbon atom to which Z² and Z⁶ are bound is aromatic.

DNA-PKcs inhibitors of formula III include compounds of formula IV or more particularly formula IVa, in which Z² is CR², Z³ is CR³, Z⁴ is CR⁴ and Z⁵ and Z⁶ are both CH:

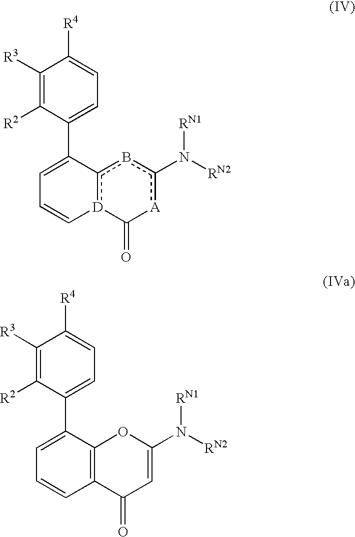

wherein A, B, D, R^{N1}, R^{N2}, R², R¹ and R⁴ are as described above. In these particular embodiments, if R³ is unsubstituted phenyl, and R^{N1} and R^{N2} form a morpholino group, R⁴ is not H.

The options for A, B and D result in compounds of the following formulae, where Ar represents the aromatic ring formed by Z², Z³, Z⁴, Z⁵ and Z⁶:

| Formula | A | B | D | Structure |
|---|---|---|---|---|
| IIIa | CH | NH | C | 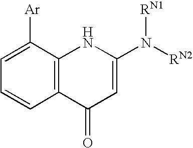 |
| IIIb | CH | N | N | 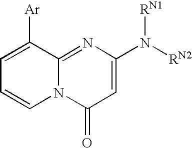 |
| IIIc | CH | O | C | 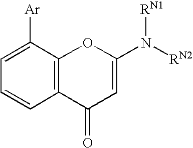 |

Preferred DNA-PKcs inhibitors of formula (III) include 8-aryl-2-morpholin-4-yl-1H-quinolin-4-one, 9-aryl-2-morpholin-4-yl-9H-pyrido[1,2-a]pyrimidin-4-one, 9-aryl-2-morpholin-4-yl-quinolizin-4-one and 5-aryl-3-morpholin-4-yl-2-benzopyran-1-one.

DNA-PKcs inhibitors having the formula (I), (II), (III), (IV) and (IVa) and their synthesis are described in more detail in WO2006/032869, WO03/024949, WO03/015790, WO2006/001379 and WO2006/001369.

The term “aromatic ring” is used herein in the conventional sense to refer to cyclic aromatic rings, that is, cyclic structures having 5 to 7 atoms in a ring with delocalised n-electron orbitals. Preferably, aromatic rings are those which meet Huckel's 4n+2 rule, ie. where the number of n-electrons is 4n+2, n representing the number of ring atoms. It is preferred that the aromatic ring has six atoms. In such a case, it is further preferred that the four atoms additional to the core moiety that make up the aromatic ring are all carbon, which yields compounds of the following general structure:

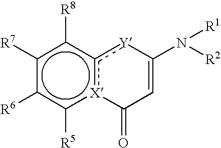

wherein X′ and Y′ are either C and O or N and N, respectively; and
where R⁵, R⁶, R⁷, and R⁸ are preferably independently selected from hydrogen, C₁₋₇ alkyl, C₃₋₂₀ heterocyclyl, C₅₋₂₀ aryl, hydroxy, C₁₋₇ alkoxy (including C₁₋₇ alkyl-C₁₋₇ alkoxy and C₃₋₂₀ aryl-C₁₋₇ alkoxy) and acyloxy or adjacent pairs of substituents (i.e. R⁵ and R⁶, R⁶ and R⁷, R⁷ and R⁸) form, together with the atoms to which they are attached, an optionally substituted aromatic or carbocyclic ring.

The fused aromatic ring represented by -A-B- may be substituted by one or more of the following groups: C₁₋₇ alkyl, C₃₋₂₀ heterocyclyl, C₅₋₂₀ aryl, hydroxy, C₁₋₇ alkoxy (including C₁₋₇ alkyl-C₁₋₇ alkoxy and C₃₋₂₀ aryl-C₁₋₇ alkoxy) and acyloxy; adjacent pairs of substituents may form, together with the atoms to which they are attached, an optionally substituted aromatic or carbocyclic ring.

The term carbocyclic ring refers to a ring formed from 5 to 7 covalently linked carbon atoms. The ring may contain one or more carbon-carbon double bonds. Examples of carbocyclic rings include cyclopentane, cyclohexane, cycloheptane, cyclopentene, cyclohexene and cycloheptene.

C₁₋₇ alkyl: The term “C₁₋₇ alkyl”, as used herein, pertains to a monovalent moiety obtained by removing a hydrogen atom from a C₁₋₇ hydrocarbon compound having from 1 to 7 carbon atoms, which may be aliphatic or alicyclic, or a combination thereof, and which may be saturated, partially unsaturated, or fully unsaturated.

Examples of saturated linear C₁₋₇ alkyl groups include, but are not limited to, methyl, ethyl, n-propyl, n-butyl, and n-pentyl (amyl).

Examples of saturated branched C₁₋₇ alkyl groups include, but are not limited to, iso-propyl, iso-butyl, sec-butyl, tert-butyl, and neo-pentyl.

Examples of saturated alicyclic C₁₋₇ alkyl groups (also referred to as “C₃₋₇ cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of unsaturated C₁₋₇ alkyl groups which have one or more carbon-carbon double bonds (also referred to as “C₂₋₇alkenyl” groups) include, but are not limited to, ethenyl (vinyl, —CH═CH₂), 2-propenyl (allyl, —CH—CH═CH₂), isopropenyl (—C(CH₃)═CH₂), butenyl, pentenyl, and hexenyl.

Examples of unsaturated C₁₋₇ alkyl groups which have one or more carbon-carbon triple bonds (also referred to as “C₂₋₇ alkynyl” groups) include, but are not limited to, ethynyl (ethinyl) and 2-propynyl (propargyl).

Examples of unsaturated alicyclic (carbocyclic) C₁₋₇ alkyl groups which have one or more carbon-carbon double bonds (also referred to as “C₃₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

C₃₋₂₀heterocyclyl: The term “C₃₋₂₀heterocyclyl”, as used herein, pertains to a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. “C₃₋₂₀” denotes ring atoms, whether carbon atoms or heteroatoms.

Examples of C₃₋₂₀ heterocyclyl groups having one nitrogen ring atom include, but are not limited to, those derived from aziridine, azetidine, pyrrolidines (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine.

Examples of C₃₋₂₀ heterocyclyl groups having one oxygen ring atom include, but are not limited to, those derived from oxirane, oxetane, oxolane (tetrahydrofuran), oxole (dihydrofuran), oxane (tetrahydropyran), dihydropyran, pyran (C₆), and oxepin. Examples of substituted C₃₋₂₀ heterocyclyl groups include sugars, in cyclic form, for example, furanoses and pyranoses, including, for example, ribose, lyxose, xylose, galactose, sucrose, fructose, and arabinose.

Examples of C₃₋₂₀ heterocyclyl groups having one sulphur ring atom include, but are not limited to, those derived from thiirane, thietane, thiolane (tetrahydrothiophene), thiane (tetrahydrothiopyran), and thiepane.

Examples of C₃₋₂₀heterocyclyl groups having two oxygen ring atoms include, but are not limited to, those derived from dioxolane, dioxane, and dioxepane.

Examples of C₃₋₂₀heterocyclyl groups having two nitrogen ring atoms include, but are not limited to, those derived from imidazolidine, pyrazolidine (diazolidine), imidazoline, pyrazoline (dihydropyrazole), and piperazine.

Examples of C₃₋₂₀heterocyclyl groups having one nitrogen ring atom and one oxygen ring atom include, but are not limited to, those derived from tetrahydrooxazole, dihydrooxazole, tetrahydroisoxazole, dihydroisoxazole, morpholine, tetrahydrooxazine, dihydrooxazine, and oxazine.

Examples of C₃₋₂₀heterocyclyl groups having one oxygen ring atom and one sulphur ring atom include, but are not limited to, those derived from oxathiolane and oxathiane (thioxane).

Examples of C₃₋₂₀ heterocyclyl groups having one nitrogen ring atom and one sulphur ring atom include, but are not limited to, those derived from thiazoline, thiazolidine, and thiomorpholine.

Other examples of C₃₋₂₀heterocyclyl groups include, but are not limited to, oxadiazine and oxathiazine.

Examples of heterocyclyl groups which additionally bear one or more oxo (═O) groups, include, but are not limited to, those derived from:
C₅ heterocyclics, such as furanone, pyrone, pyrrolidone (pyrrolidinone), pyrazolone (pyrazolinone), imidazolidone, thiazolone, and isothiazolone;
C₆ heterocyclics, such as piperidinone (piperidone), piperidinedione, piperazinone, piperazinedione, pyridazinone, and pyrimidinone (e.g., cytosine, thymine, uracil), and barbituric acid;
fused heterocyclics, such as oxindole, purinone (e.g., guanine), benzoxazolinone, benzopyrone (e.g., coumarin);
cyclic anhydrides (—C(═O)—O—C(═O)— in a ring), including but not limited to maleic anhydride, succinic anhydride, and glutaric anhydride;
cyclic carbonates (—O—C(═O)—O— in a ring), such as ethylene carbonate and 1,2-propylene carbonate;
imides (—C(═O)—NR—C(═O)— in a ring), including but not limited to, succinimide, maleimide, phthalimide, and glutarimide;
lactones (cyclic esters, —O—C(═O)— in a ring), including, but not limited to, β-propiolactone, γ-butyrolactone, δ-valerolactone (2-piperidone), and ε-caprolactone;
lactams (cyclic amides, —NR—C(═O)— in a ring), including, but not limited to, β-propiolactam, γ-butyrolactam (2-pyrrolidone), δ-valerolactam, and ε-caprolactam;
cyclic carbamates (—O—C(═O)—NR— in a ring), such as 2-oxazolidone;
cyclic ureas (—NR—C(═O)—NR— in a ring), such as 2-imidazolidone and pyrimidine-2,4-dione (e.g., thymine, uracil).

C₅₋₂₀ aryl: The term “C₅₋₂₀ aryl”, as used herein, pertains to a monovalent moiety obtained by removing a hydrogen atom from an aromatic ring atom of a C₅₋₂₀aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups”, in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), naphthacene (C₁₈), and pyrene (C₁₆).

Examples of aryl groups which comprise fused rings, one of which is not an aromatic ring, include, but are not limited to, groups derived from indene and fluorene.

Alternatively, the ring atoms may include one or more heteroatoms, including but not limited to oxygen, nitrogen, and sulphur, as in “heteroaryl groups”. In this case, the group may conveniently be referred to as a “C₅₋₂₀ heteroaryl”, group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

Examples of C₅₋₂₀ heterocyclic groups (some of which are C₅₋₂₀ heteroaryl groups) which comprise fused rings, include, but are not limited to, Cg heterocyclic groups derived from benzofuran, isobenzofuran, indole, isoindole, purine (e.g., adenine, guanine), benzothiophene, benzimidazole; C₁₀ heterocyclic groups derived from quinoline, isoquinoline, benzodiazine, pyridopyridine, quinoxaline; C₁₋₃ heterocyclic groups derived from carbazole, dibenzothiophene, dibenzofuran; C₁₄ heterocyclic groups derived from acridine, xanthene, phenoxathiin, phenazine, phenoxazine, phenothiazine.

The above C₁₋₇ alkyl, C₃₋₂₀ heterocyclyl, and C₅₋₂₀ aryl groups, whether alone or part of another substituent, may themselves optionally be substituted with one or more groups selected from themselves and the additional substituents listed below.

### Halo: —F, —Cl, —Br, and —I.

### Hydroxy: —OH.

Ether: —OR, wherein R is an ether substituent, for example, a C₁₋₇ alkyl group (also referred to as a C₁₋₇ alkoxy group, discussed below), a C₃₋₂₀heterocyclyl group (also referred to as a C₃₋₂₀ heterocyclyloxy group), or a C₅₋₂₀ aryl group (also referred to as a C₅₋₂₀aryloxy group), preferably a C₁₋₇ alkyl group.
C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).
Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.
Imino (imine): ═NR, wherein R is an imino substituent, for example, hydrogen, C₁₋₇ alkyl group, a C₃₋₂₀heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.
Formyl (carbaldehyde, carboxaldehyde): —C(═O)H.
Acyl (keto): —C(═O)R, wherein R is an acyl substituent, for example, a C₁₋₇alkyl group (also referred to as C₁₋₇ alkylacyl or C₁₋₇ alkanoyl), a C₃₋₂₀ heterocyclyl group (also referred to as C₃₋₂₀heterocyclylacyl), or a C₅₋₂₀aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).
Carboxy (carboxylic acid): —COOH.
Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.
Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.
Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(—O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.
Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC (═O)CH₃, —NHC(═O)CH₂CH₃, and —NHC(═O) Ph. R¹ and R² may together form a cyclic structure, as in, for example, succinimidyl, maleimidyl and phthalimidyl:

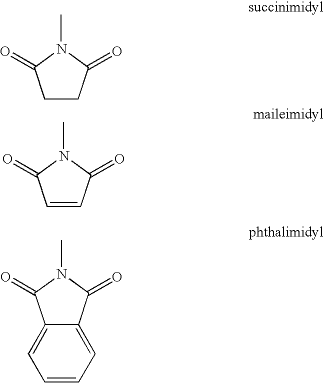

Acylureido: —N(R¹)C(O)NR²C(O)R³ wherein R¹ and R² are independently ureido substituents, for example, hydrogen, a C₁₋₇ alkyl group, a C₃₋₂₀ heterocyclyl group, or a C₅₋₂₀ aryl group, preferably hydrogen or a C₁₋₇ alkyl group. R³ is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.
Carbamate: —NR¹—C(O)—OR² wherein R¹ is an amino substituent as defined for amino groups and R² is an ester group as defined for ester groups. Examples of carbamate groups include, but are not limited to, —NH—C(O)—O-Me, —NMe-C(O)—O-Me, —NH—C(O)—O-Et, —NMe-C(O)—O-t-butyl, and —NH—C(O)—O-Ph.
Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.
Tetrazolyl: a five membered aromatic ring having four nitrogen atoms and one carbon atom,

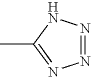

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.
Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group.
Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. An example of an amidine group is —C(═NH)NH₂.
Carbazoyl (hydrazinocarbonyl): —C(O)—NN—R¹ wherein R¹ is an amino substituent as defined for amino groups. Examples of azino groups include, but are not limited to, —C(O)—NN—H, —C(O)—NN-Me, —C(O)—NN-Et, —C(O)—NN-Ph, and —C(O)—NN—CH₂-Ph.

### Nitro: —NO₂.

### Nitroso: —NO.

### Azido: —N₃.

Cyano (nitrile, carbonitrile): —CN.

### Isocyano: —NC.

### Cyanato: —OCN.

### Isocyanato: —NCO.

Thiocyano (thiocyanato): —SCN.
Isothiocyano (isothiocyanato): —NCS.
Sulfhydryl (thiol, mercapto): —SH.
Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.
Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.
Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃, —S(═O)₂C₄F₉ (nonaflyl), —S(═O)₂CH₂CF₃ (tresyl), —S(═O)₂Ph (phenylsulfonyl), 4-methylphenylsulfonyl (tosyl), 4-bromophenylsulfonyl (brosyl), and 4-nitrophenyl (nosyl).
Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.
Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ and —OS(═O)₂CH₂CH₃.
Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.
Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.
Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.
Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.
Sulfamyl: —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.
Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅. A special class of sulfonamino groups are those derived from sultams—in these groups one of R¹ and R is a C₅₋₂₀ aryl group, preferably phenyl, whilst the other of R¹ and R is a bidentate group which links to the C₅₋₂₀ aryl group, such as a bidentate group derived from a C₁₋₇ alkyl group. Examples of such groups include, but are not limited to:

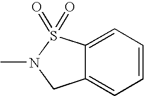

### 2,3-dihydro-benzo[d]isothiazole-1,1-dioxide-2-yl

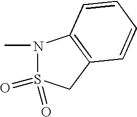

### 1,3-dihydro-benzo[c]isothiazole-2,2-dioxide-1-yl

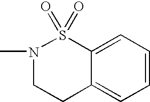

### 3,4-dihydro-2H-benzo[e][1,2]thiazine-1,1-dioxide-2-yl

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.
Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O) (OCH₂CH₃)—N(CH₃)₂, —OP(═O) (OCH₂CH₃)—N (i-Pr)₂, and —OP(═O) (OCH₂CH₂CN)—N (i-Pr)₂.
C₁₋₅ Alkylene: The term “C₁₋₅ alkylene”, as used herein, pertains to a bidentate moiety obtained by removing two hydrogen atoms, either both from the same carbon atom, or one from each of two different carbon atoms, of an aliphatic linear hydrocarbon compound having from 1 to 5 carbon atoms (unless otherwise specified), which may be saturated, partially unsaturated, or fully unsaturated. Thus, the term “alkylene” includes the sub-classes alkenylene, alkynylene, etc., discussed below.

Examples of saturated C₁₋₅ alkylene groups include, but are not limited to, —(CH₂)ₙ— where n is an integer from 1 to 5, for example, —CH₂— (methylene), —CH₂CH₂— (ethylene), —CH₂CH₂CH₂— (propylene), and —CH₂CH₂CH₂CH₂— (butylene).

Examples of partially unsaturated C₁₋₅ alkylene groups include, but is not limited to, —CH═CH— (vinylene), —CH═CH—CH₂—, —CH₂—CH═CH₂—, —CH═CH—CH₂—CH₂-, —CH═CH—CH₂—CH₂—CH₂—, —CH═CH—CH═CH— and —CH═CH—CH═CH—CH₂—.

The substituent groups listed above may be substituents on an alkylene group.

In many cases, substituents may themselves be substituted. For example, a C₁₋₇ alkoxy group may be substituted with, for example, a C₁₋₇ alkyl (also referred to as a C₁₋₇ alkyl-C₁₋₇alkoxy group), for example, cyclohexylmethoxy, a C₃₋₂₀ heterocyclyl group (also referred to as a C₅₋₂₀ aryl-C₁₋₇ alkoxy group), for example phthalimidoethoxy, or a C₅₋₂₀ aryl group (also referred to as a C₅₋₂₀aryl-C₁₋₇alkoxy group), for example, benzyloxy.

Included in the above are the well-known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

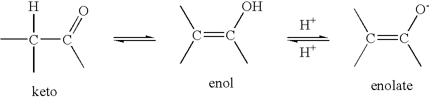

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts”, J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃₁—NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO$).

For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₇ alkyl (e.g.-Me, -Et); C₁₋₇ aminoalkyl (e.g. aminoethyl; 2-(N,N-diethylamino)ethyl; 2-(4-morpholino) ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Preferred DNA-PKcs inhibitors include 8-aryl-2-morpholin-4-yl-1-benzopyran-4-one (WO2006/032869), 8-aryl-2-morpholin-4-yl-1H-quinolin-4-one, 9-aryl-2-morpholin-4-yl-9H-pyrido[1,2-a]pyrimidin-4-one, 9-aryl-2-morpholin-4-yl-quinolizin-4-one or 5-aryl-3-morpholin-4-yl-2-benzopyran-1-one (all US60/671,830, WO2006/001379 and WO2006/001369), where the aryl group is described as dibenzothiophenyl, dibenzofuranoyl in nature.

In some preferred embodiments, 2-(4-Ethyl-piperazin-1-yl)-N-[4-(2-morpholin-4-yl-4-oxo-4H-1-benzopyran-8-yl)-dibenzothiophen-1-yl]-acetamide (KU-0060648: WO2006/032869: formula V) is employed as a DNA-PKcs inhibitor:

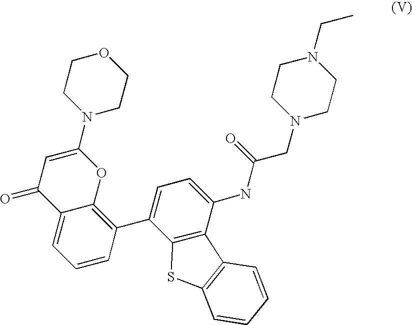

Another class of suitable DNA-PKcs inhibitors includes peptide fragments of DNA-PKcs. Peptide fragments may be generated wholly or partly by chemical synthesis using the published sequences of the components. Peptide fragments can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Other compounds for inhibiting DNA-PKcs are based on modelling the 3-dimensional structure of DNA-PKcs and using rational drug design to provide candidate compounds with particular molecular shape, size and charge characteristics. A candidate inhibitor, for example, may be a “functional analogue” of a peptide fragment or other compound which inhibits the component. A functional analogue has the same functional activity as the peptide or other compound in question, i.e. it may interfere with the interactions or activity of the DNA repair pathway component. Examples of such analogues include chemical compounds which are modelled to resemble the three dimensional structure of the component in an area which contacts another component, and in particular the arrangement of the key amino acid residues as they appear.

Another class of suitable DNA-PKcs inhibitors includes nucleic acid encoding part or all of the amino acid sequence of DNA-PKcs, or the complement thereof, which inhibit activity or function by down-regulating production of active DNA-PKcs polypeptide. For instance, expression of DNA-PKcs may be inhibited using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman and Ulman, Chemical Reviews, 90:543-584, (1990) and Crooke, Ann. Rev. Pharmacol. Toxicol. 32:329-376, (1992).

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16, 12:3675-3684; and Voinnet & Baulcombe (1997) Nature 389: pg 553). Double stranded RNA (dsRNA) has been found to be even more effective in gene silencing than both sense or antisense strands alone (Fire A. et al Nature 391, (1998)). dsRNA mediated silencing is gene specific and is often termed RNA interference (RNAi). RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore P. D. Nature Structural Biology, 8, 9, 746-750, (2001). RNAi may also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologous genes in a wide range of mammalian cell lines (Elbashir S M. et al. Nature, 411, 494-498, (2001)).

Another possibility is that nucleic acid is used which on transcription produces a ribozyme, able to cut nucleic acid at a specific site—thus also useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

A DNA damaging chemotherapeutic agent is preferably a compound which induces DNA DSBs in cellular DNA. Many suitable compounds are known in the art for use in the treatment of cancer, including, for example, bleomycin and inhibitors of topoisomerase I and II activity, such as doxorubicin, etoposide and members of the tecan family e.g. irinotecan, topotecan, rubitecan. Compounds that indirectly induce DSBs through the disruption of DNA synthesis, for example, gemcitabine, or through the alkylation of DNA, for example, temozolomide and DTIC (dacarbazine), or through the introduction of a bulky adduct, for example platinum agents like cisplatin, oxaliplatin and carboplatin, may also be used. Other suitable chemotherapeutic agents include yondelis. Derivatives or salts or combinations any of these compounds may also be used.

Suitable combinations of compounds that may be used as DNA damaging chemotherapeutic agents in accordance with the invention are shown in Table 8.

In some preferred embodiments, etoposide or doxorubicin may be employed.

Preferably, the DNA damaging chemotherapeutic agent is used in a dosage or formulation that, in the absence of the DNA-PKcs inhibitor, is not lethal to normal cells. Suitable dosages and regimens for DNA damaging chemotherapeutic agents are well known to medical practitioners.

The use of irradiation to induce DNA damage in cancer cells is well known in the art and any suitable technique may be used to irradiate cancer cells with an ATM deficient phenotype as described herein.

Irradiation includes external beam therapy, such as X-rays, gamma rays and electrons. Suitable regimes include fractionated palliative and curative regimes involving accelerated- and hyper-fractionation as appropriate and all geometric forms, conventional, 3D, 3D conformal, IMRT (intensity modulated radiotherapy), 4D and adaptive radiotherapy. (Bucci M K et al [2005] CA Cancer J Clin 55; 117-134, Haustermans et al (2004) Rays 29(3):231-6).

Irradiation includes local/targeted therapies, such as radio active seeds or wires surgically implanted as part of a brachytherapy regime (Dale at al [1998] B J Radiol 71; 465-483); radioimmunotherapy, where a radioactive emitter is linked to an immunologic molecule such as a monoclonal antibody e.g. ibritumomab (Zevalin) (Blum K A, Bartlett N L [2004] Expert Opin Biol Ther. 4(8):1323-31); and non-immunological targeting such as radioactive microspheres delivered by injection e.g. SIR-Spheres® (Ho S et al (2001) Journal of Nuclear Medicine 42(10):1587-1589). Non-immunological targeting may also be accomplished with targeted peptide receptor therapy. For example, radiolabelled somatostatin analogues (¹¹¹In-Octreotide, ⁹⁰Y-OctreoTher™, ¹⁷⁷Lu-Octreotate) or other peptide ligands, such as Bombesin and NPY(y₁)analogues (Krenning et al [2004] Ann NY Acad Sci. 1014(2): 234-245)

Methods of the invention may comprise administering a DNA-PKcs inhibitor to an individual. Administration may be simultaneously or sequentially to the administration of a DNA damaging chemotherapeutic agent or irradiation therapy. In some embodiments, this occurs subsequent to having identified the individual as having a cancer condition which has an ATM deficient phenotype.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Pharmaceutical compositions comprising a DNA-PKcs inhibitor, for example an inhibitor admixed together with one or more pharmaceutically acceptable carriers, excipients, buffers, adjuvants, stabilisers, or other materials, as described herein, may be used in the methods described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The inhibitor or pharmaceutical composition comprising the inhibitor may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Appropriate dosages of the active compounds, and compositions comprising the active compounds, can vary from patient to patient. Determining the optimal dosage will generally involve the balancing of the level of therapeutic benefit against any risk or deleterious side effects of the treatments. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action that achieve the desired effect without causing substantial harmful or deleterious side-effects.

The methods described herein may also be useful in determining the susceptibility of a cancer condition to cancer therapy. For example, the identification of a cancer cell obtained from an individual as having an ATM deficient phenotype may be indicative that the individual has a cancer condition which is susceptible to treatment with a combination of a DNA-PKcs inhibitor and a DNA damaging chemotherapeutic agent or irradiation. Techniques for determining whether a cancer cell has an ATM deficient phenotype are described in more detail above.

A cancer cell obtained from an individual may be comprised within a biopsy or sample which has been previously isolated or removed from the individual.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described above and tables described below.

Table 1 shows etoposide and doxorubicin doses used to demonstrate chemopotentiation,

Table 2 shows the effect of etoposide in combination with the DNA-PKcs inhibitor KU-0060648 on pEBS7 (ATM-null) cells.

Table 3 shows the effect of etoposide in combination with the DNA-PKcs inhibitor KU-0060648 on pEBS7-YZ (ATM+/+ cells).

Table 4 shows the effect of doxorubicin in combination with the DNA-PKcs inhibitor KU-0060648 on pEBS7 (ATM-null cells).

Table 5 shows the effect of doxorubicin in combination with the DNA-PKcs inhibitor KU-0060648 on pEBS7-YZ (ATM+/+ cells).

Table 6 shows the effect of ionising radiation in combination with the DNA-PKcs inhibitor KU-0060648 on % survival of ATM-null (pEBS7) and ATM+/+ (pEBS7-YZ) cells.

Table 7 shows the potentiation of chemotherapy and ionising radiation in combination with the DNA-PKcs inhibitor KU-0060648 on ATM-null (pEBS7) and ATM+/+ (pEBS7-YZ) cells

Table 8 shows combinations of one or more DNA damaging chemotherapeutic agents which may be used in therapy.

Table 9 shows alterations in the ATM sequence which are deleterious to ATM function which are known in cancer patients who do not have Ataxia-Telangiectasia (from the Ataxia-Telangiectasia Mutation Database updated 27 May 2004)

### Materials and Methods

### Cell Lines

The A-T fibroblast AT221JE-T cells (pEBS7, herein referred to as ATM-null cells) and the trans-complemented ATM+/+ (pEBS7-YZ5, herein referred to as ATM-competent cells) were grown in DMEM+20% FBS PSG and 100 ug/ml hygromycin. These cells are immortalised fibroblasts from an A-T patient which were transfected with pEBS7-YZ5 vector coding for full length ATM and the hygromycin resistance marker or pEBS7 vector, coding for the hygromycin resistance marker only. (Ziv Y et al (1997) Oncogene 15, 159-67)

In order to demonstrate chemopotentiation, cells were plated into 96 well assay plates at a density of 2×10⁴ cells per ml in a volume of 90 ul. Following a 4-hour incubation to allow cell adherence, 5 μl DNA-PKcS inhibitor KU-0060648 (final concentration 0.5 μM) or DMSO/PBS equivalent was added. After a further 1 hour incubation, 5 μl of chemotherapeutic drug was added per well (see table 1). Drugs were removed after 16 hours and 150 μl fresh medium added per well. After 24h, one plate for each cell line was fixed and stained to account for background differences between the lines. Five days after seeding, the medium was aspirated from the wells of the remaining assay plates and 100 μl ice cold 10% TCA added for 30 minutes at 4° C. The wells were then washed four times with tap water and 50 μl 0.4% sulforhodamine B in 1% acetic acid added per well. After 15 minutes incubation at room temperature, excess stain was washed from the wells with 1% acetic acid. 100 μl mM Tris pH6.8 was then added per well, the plates shaken to resuspend the stain and the absorbance at 564 nm determined.

To show radio-potentiation, 2×10⁵ cells per well were seeded into 6 well cluster plates. One plate was used for each dose of irradiation. After allowing the cells to adhere for 4 hours, the cells were dosed with a final concentration of 0.5 μM of the DNA-PKcs inhibitor KU-0060648 or equivalent DMSO/PBS control. After further incubation for 1 hour, the cells were irradiated with 0, 0.5, 1, 2 and 4 Grays. After 16 hours incubation, the cells were washed with 1 ml PBS and 500 μl trypsin EDTA added for 5 minutes at 37° C. 1.5 ml fresh medium was then added and the cell density in the untreated (0 Gy) wells determined. All subsequent calculations for seeding densities were performed according to this cell count. Cells were then replated into 2 mls fresh medium in 6 well cluster plates at the appropriate cell densities. After 6 days' incubation, the medium was removed by aspiration and cell colonies stained using 400 μl per well Giemsa stain for 20 minutes. Excess stain was washed from the plates with tap water and the plates left to air-dry. The number of colonies per well was then counted using Colcount software.

### DNA-PKcs Assay

DNA-PKcs activity was determined in samples of tumour harvested in either preclinical or clinical studies. Tumour samples were homogenised with a mechanical homogeniser for one minute, on ice, in 3 volumes (w/v) of extraction buffer (450 mM NaCl, 20 mM HEPES pH7.4, 50 mM NaF, 1 mM NaVO4, 25% (v/v) Glycerol, 200 μM EDTA, 500 μM DTT, plus protease inhibitors [Roche]). The samples were then subjected to three freeze-thaw cycles and cell debris removed by centrifugation at 13,000 rpm for 30 minutes at 4° C. 50 μg of protein extract was then used to determine DNA-PKcs activity against a p53 fusion protein substrate containing the serine-15 phosphorylation site (Veuger et al (2003) Cancer Research 63, 6008-6015). Briefly, the assay reaction contained 50 μg tumour extract, 1 ng DNA, 50 μM ATP and 1 μg p53 substrate. Negative controls contained no DNA. The reaction was stopped by the addition of 6M guanidine, and the amount of phosphorylation on the serinel5 site of the substrate was determined by ELISA using a phospho-serinel5 p53 specific antibody (Cell signalling technology) and a luminescent readout.

### Results

The inhibition of DNA-PKcs and the therapeutic potentiation of DSB inducing chemotherapies, doxorubicin and etoposide, were investigated in ATM-null pEBS7 and ATM-competent pEBS7-YZ5 cells. Although not tumour derived, the ATM-null pEBS7 cells are representative of ATM dysfunctional tumour cells.

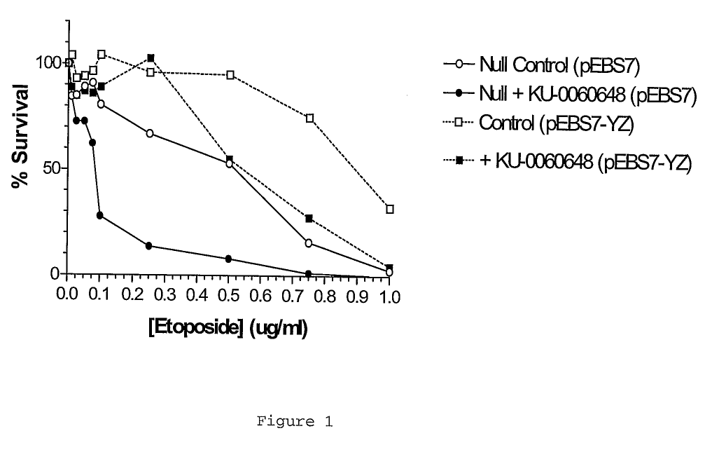

The effect of etoposide in combination with the DNA-PKcs inhibitor KU-0060648 on ATM-competent and ATM-null cells was assessed. The data are shown tabulated and graphically in Table 2, Table 3 and FIG. 1.

Etoposide was observed to be significantly potentiated by the KU-0060648 in ATM-null but not ATM-competent cells.

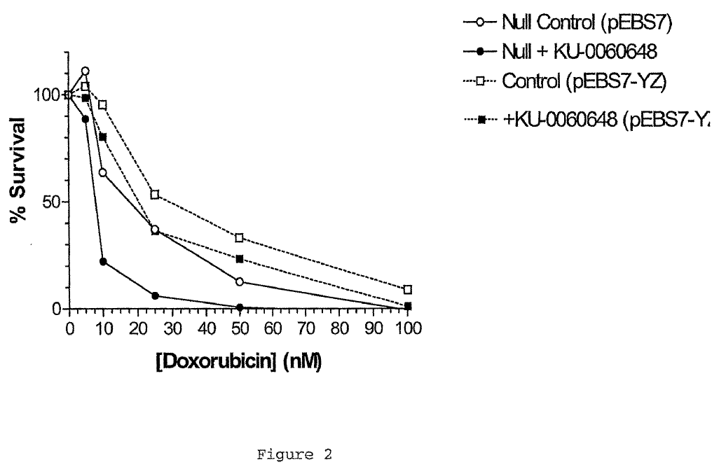

The effect of doxorubicin in combination with the DNA-PKcs inhibitor KU-0060648 on ATM-competent and ATM-null cells was assessed. The data are shown tabulated and graphically in Table 4, Table 5 and FIG. 2.

KU-0060648 was found to significantly potentiate the effect of doxorubicin in ATM-null cells but not ATM-competent cells.

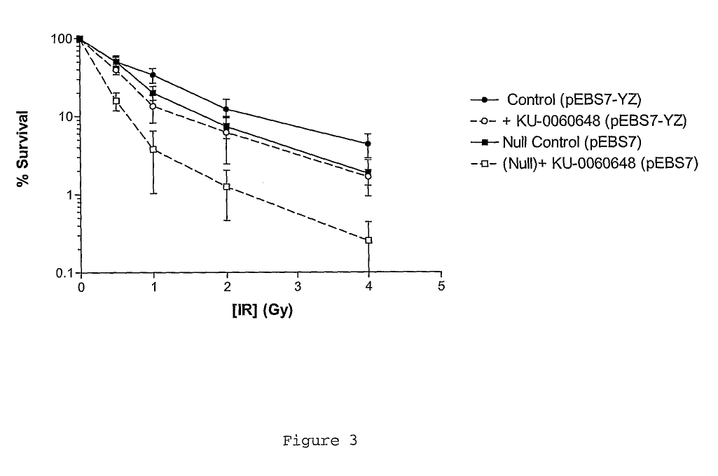

The effect of ionising radiation in combination with the DNA-PKcs inhibitor KU-0060648 on ATM-competent and ATM-null cells was assessed. The data are shown tabulated and graphically in Table 6 and FIG. 3.

KU-0060648 was found to significantly potentiate the effect of irradiation in ATM-null cells but not ATM-competent cells.

The potentiation of etoposide, doxorubicin and ionising radiation, was observed in DNA-PKcs inhibited ATM-null (pEBS7) cells. However, increased potentiation was not seen in the matched ATM-competent cell line (pEBS7-YZ), (Table 7).

In table 7, PF₅₀ is the potentiation factor at 50% cell kill. This is derived from the chemotherapeutic concentration giving 50% cell kill in the absence of DNA-PKcs inhibitor, divided by the chemotherapeutic concentration giving 50% cell kill in the presence of DNA-PKcs inhibitor. Dose modification ratio (DMR) is the ratio of the number of cells that survive a single 2 Gy treatment and the number of cells that survive a single 2 Gy treatment in combination with a given concentration of DNA-PKcs inhibitor.

TABLE 1

| [Etoposide] | [Doxorubicin] |
|---|---|
| (μg/ml) | (nM) |
| 0.0 | 0.0 |
| 0.01 | 0.25 |
| 0.025 | 0.5 |
| 0.05 | 1.0 |
| 0.075 | 2.5 |
| 0.1 | 5.0 |
| 0.25 | 10.0 |
| 0.5 | 25.0 |
| 0.75 | 50.0 |
| 1.0 | 100.0 |
| 2.5 | 250.0 |
| 5.0 | 500.0 |

TABLE 2

| | | | | | | | | | | | | | |
|---|---|---|---|---|---|---|---|---|---|---|---|---|---|
| Con | [Etoposide] | 0 | 0.01 | 0.025 | 0.05 | 0.075 | 0.1 | 0.25 | 0.5 | 0.75 | 1 | 2.5 | 5 |
| | SD | 0.27 | 0.24 | 0.14 | 0.22 | 0.26 | 0.17 | 0.09 | 0.13 | 0.12 | 0.04 | 0.03 | 0.01 |
| | Avg | 1.63 | 1.38 | 1.38 | 1.45 | 1.48 | 1.31 | 1.09 | 0.86 | 0.26 | 0.04 | 0.02 | −0.03 |
| | % survival | 100 | 84.6 | 84.9 | 89.0 | 90.8 | 80.6 | 66.8 | 53.0 | 15.8 | 2.5 | 1.2 | −1.6 |
| | SD | 16.8 | 14.7 | 8.6 | 13.4 | 15.7 | 10.3 | 5.6 | 8.0 | 7.4 | 2.6 | 1.9 | 0.6 |
| KU-0060648 | [Etoposide] | 0 | 0.01 | 0.025 | 0.05 | 0.075 | 0.1 | 0.25 | 0.5 | 0.75 | 1 | 2.5 | 5 |
| | SD | 0.32 | 0.09 | 0.11 | 0.12 | 0.08 | 0.06 | 0.06 | 0.06 | 0.02 | 0.01 | 0.01 | 0.02 |
| | Avg | 1.61 | 1.36 | 1.17 | 1.17 | 1.00 | 0.45 | 0.22 | 0.13 | 0.02 | −0.01 | −0.02 | −0.03 |
| | % survival | 100 | 84.7 | 72.7 | 72.7 | 62.3 | 27.9 | 13.6 | 7.9 | 1.2 | −0.4 | −1.5 | −1.7 |
| | SD | 19.7 | 5.3 | 6.7 | 7.7 | 4.7 | 4.0 | 3.6 | 3.9 | 1.3 | 0.5 | 0.8 | 0.9 |

TABLE 3

| | | | | | | | | | | | | | |
|---|---|---|---|---|---|---|---|---|---|---|---|---|---|
| Con | [Etoposide] | 0 | 0.01 | 0.025 | 0.05 | 0.075 | 0.1 | 0.25 | 0.5 | 0.75 | 1 | 2.5 | 5 |
| | SD | 0.17 | 0.09 | 0.02 | 0.03 | 0.10 | 0.12 | 0.03 | 0.10 | 0.14 | 0.11 | 0.10 | 0.13 |
| | Avg | 1.67 | 1.74 | 1.56 | 1.58 | 1.62 | 1.75 | 1.60 | 1.59 | 1.26 | 0.54 | 0.65 | 0.36 |
| | % survival | 100 | 103.9 | 93.1 | 94.1 | 96.6 | 104.2 | 95.8 | 95.0 | 75.1 | 32.4 | 39.0 | 21.7 |
| | SD | 10.4 | 5.4 | 1.5 | 1.7 | 6.0 | 7.2 | 1.9 | 6.0 | 8.4 | 6.4 | 5.8 | 7.9 |
| KU-0060648 | [Etoposide] | 0 | 0.01 | 0.025 | 0.05 | 0.075 | 0.1 | 0.25 | 0.5 | 0.75 | 1 | 2.5 | 5 |
| | SD | 0.18 | 0.12 | 0.15 | 0.13 | 0.08 | 0.07 | 0.10 | 0.10 | 0.06 | 0.04 | 0.03 | 0.12 |
| | Avg | 1.79 | 1.59 | 1.52 | 1.56 | 1.54 | 1.59 | 1.84 | 0.99 | 0.49 | 0.08 | 0.10 | 0.15 |
| | % survival | 100 | 88.8 | 84.9 | 87.2 | 86.1 | 88.9 | 102.7 | 55.0 | 27.6 | 4.5 | 5.7 | 8.6 |
| | SD | 10.2 | 6.8 | 8.6 | 7.3 | 4.5 | 3.8 | 5.8 | 5.4 | 3.5 | 2.1 | 1.8 | 6.9 |

TABLE 4

| | | | | | | | | | | | | | |
|---|---|---|---|---|---|---|---|---|---|---|---|---|---|
| Con | Doxorubicin | 0 | 0.01 | 0.025 | 0.05 | 0.075 | 0.1 | 0.25 | 0.5 | 0.75 | 1 | 2.5 | 5 |
| | SD | 0.50 | 0.06 | 0.07 | 0.04 | 0.13 | 0.08 | 0.14 | 0.11 | 0.09 | 0.04 | 0.02 | 0.01 |
| | Avg | 1.30 | 1.46 | 1.53 | 1.39 | 1.47 | 1.45 | 0.83 | 0.49 | 0.16 | −0.01 | −0.03 | −0.05 |
| | % survival | 100 | 111.8 | 117.0 | 106.4 | 113.1 | 111.2 | 63.8 | 37.3 | 12.6 | −0.9 | −2.6 | −3.6 |
| | SD | 38.1 | 4.4 | 5.4 | 3.1 | 10.2 | 6.1 | 10.4 | 8.1 | 7.2 | 3.0 | 1.9 | 0.5 |
| KU-0060648 | Doxorubicin | 0 | 0.01 | 0.025 | 0.05 | 0.075 | 0.1 | 0.25 | 0.5 | 0.75 | 1 | 2.5 | 5 |
| | SD | 0.08 | 0.11 | 0.40 | 0.28 | 0.26 | 0.27 | 0.11 | 0.05 | 0.01 | 0.01 | 0.02 | 0.03 |
| | Avg | 1.42 | 1.53 | 1.23 | 1.15 | 1.17 | 1.26 | 0.31 | 0.09 | 0.01 | −0.03 | −0.03 | −0.01 |
| | % survival | 100 | 107.9 | 87.0 | 81.3 | 82.3 | 88.7 | 22.1 | 6.2 | 0.6 | −2.2 | −2.0 | −0.8 |
| | SD | 5.5 | 7.5 | 28.5 | 19.7 | 18.7 | 19.2 | 8.0 | 3.6 | 0.7 | 0.4 | 1.5 | 2.0 |

TABLE 5

| | | | | | | | | | | | | | |
|---|---|---|---|---|---|---|---|---|---|---|---|---|---|
| Con | [Doxorubicin] | 0 | 0.01 | 0.025 | 0.05 | 0.075 | 0.1 | 0.25 | 0.5 | 0.75 | 1 | 2.5 | 5 |
| | SD | 0.07 | 0.15 | 0.10 | 0.19 | 0.11 | 0.10 | 0.15 | 0.06 | 0.04 | 0.04 | 0.02 | 0.02 |
| | Avg | 1.62 | 1.43 | 1.45 | 1.42 | 1.53 | 1.69 | 1.55 | 0.87 | 0.54 | 0.14 | −0.03 | −0.04 |
| | % survival | 100 | 88.3 | 89.4 | 87.3 | 94.1 | 103.9 | 95.3 | 53.4 | 33.2 | 8.8 | −1.7 | −2.6 |
| | SD | 4.5 | 9.1 | 6.5 | 11.6 | 6.6 | 6.1 | 9.3 | 3.8 | 2.7 | 2.4 | 1.4 | 1.2 |
| KU-0060648 | [Doxorubicin] | 0 | 0.01 | 0.025 | 0.05 | 0.075 | 0.1 | 0.25 | 0.5 | 0.75 | 1 | 2.5 | 5 |
| | SD | 0.05 | 0.15 | 0.03 | 0.12 | 0.03 | 0.13 | 0.13 | 0.10 | 0.09 | 0.01 | 0.01 | 0.04 |
| | Avg | 1.62 | 1.50 | 1.56 | 1.67 | 1.53 | 1.60 | 1.30 | 0.59 | 0.38 | 0.02 | −0.03 | −0.03 |
| | % survival | 100 | 92.3 | 96.1 | 102.9 | 94.1 | 98.5 | 80.4 | 36.6 | 23.4 | 1.1 | −2.1 | −1.9 |
| | SD | 3.2 | 9.1 | 1.9 | 7.7 | 1.9 | 8.2 | 7.9 | 6.0 | 5.6 | 0.9 | 0.5 | 2.7 |

TABLE 6

| | Control | +KU-0060648 | | +KU- |
|---|---|---|---|---|
| | (ATM- | (ATM- | Control | 0060648 |
| [IR] | competent | competent | (ATM-null | (ATM-null |
| (Gy) | pEBS7-YZ) | pEBS7-YZ) | pEBS7) | pEBS7) |
| 0 | 100.0 | 100.0 | 100.0 | 100.0 |
| 0.5 | 50.0 | 39.3 | 50.3 | 15.8 |
| 1 | 34.1 | 13.5 | 20.1 | 3.8 |
| 2 | 12.3 | 6.2 | 7.4 | 1.3 |
| 4 | 4.4 | 1.7 | 1.8 | 0.3 |

TABLE 7

| | | |
|---|---|---|
| | pEBS7-YZ | pEBS7 |
| Damaging | (ATM-competent) | (ATM-null) |

| Agent | PF50 | 2Gy DMR | PF50 | 2Gy DMR |
|---|---|---|---|---|
| Etoposide | 1.7 | | 6.5 | |
| Doxorubicin | 1.5 | | 2.3 | |
| Ionising | | 2.0 | | 5.7 |
| radiation | | | | |

TABLE 8
Bleomycin

| | |
|---|---|
| ABVD | doxorubicin + Bleo + vinblastine + dacarbazine |
| ABV | doxorubicin + Bleo + vinblastine |
| BCD | Bleomycin + cyclophosphamide + dactinomycin |
| BEACOPP | bleomycin + etoposide + doxorubicin + |
| | cyclophosphamide + vincristine + procarbazine + |
| | prednisone |
| BEC | cisplatin + epirubicin + Bleo |
| BEP | Bleo + etoposide + cisplatin |
| BIP | Bleo + Cisplatin + ifosfamide |
| CMB | cisplatin + methotrexate + bleomycin - or - bleo + |
| | methotrexate + folinic acid + cisplatin |
| CHOP-B | cyclophosphamide + doxorubicin + vincristine + |
| | prednisone + bleo |
| COPP-ABVD | cyclophosphamide + vincristine + procarbazine + |
| alternating | prednisone + doxorubicin + bleomycin + |
| | vinblastine + dacarbazine |
| JEB | etoposide + carboplatin + Bleo |
| MOPP/ABV | Nitrogen mustard + vincristine + procarbazine + |
| hybrid | prednisone + doxorubicin + bleomycin + vinblastine |
| PACEBOM | prednisolone + doxorubicin + cyclophosphamide + |
| | etoposide + bleomycin + vincristine + methotrexate |
| POMMB/ACE | vincristine + methotrexate + folinic acid + |
| | bleomycin + cisplatin then dactinomycin + |
| | cyclophosphamide + etoposide |
| PBF | cisplatin + Bleo + 5-FU |
| PVB | cisplatin + vinblastine + Bleo |
| Stanford V | doxorubicin + vinblastine + nitrogen mustard + |
| | vincristine + bleomycin + etoposide + prednisone |

Etoposide (Vepesid ®, Etopophos ®, Epsin ®

| | |
|---|---|
| ADE | daunorubicin + cytarabine + etoposide |
| BEACOPP | bleomycin + etoposide + doxorubicin + |
| | cyclophosphamide + vincristine + procarbazine + |
| | prednisone |
| BEC | cisplatin + epirubicin + Bleo |
| BEP | Bleo + etoposide + cisplatin |
| CAE | cyclophosphamide + doxorubicin + etoposide |
| CDEC | cisplatin + doxorubicin + etoposide + |
| | cyclophosphamide |
| CE | carboplatin + etoposide |
| CEC | carboplatin + etoposide + cyclophosphamide |
| CEI | carboplatin + etoposide + ifosfamide |
| CEM-TBI | carboplatin + etoposide + melphalan + total body |
| | irradiation |
| ChlVPP/EVA | chlorambucil + vincristine + procarbazine + |
| hybrid | etoposide + prednisolone + doxorubicin + |
| | vinblastine |
| CHOEP | cyclophosphamide + doxorubicin + vincristine + |
| | prednisone + etoposide |
| CIDE | cisplatin + ifosfamide + doxorubicin + etoposide |
| Dexa-BEAM | dexamethasone + BCNU + etoposide + cytarabine + |
| | melphalan |
| EP | cisplatin + etoposide |
| EPIC | etoposide + prednisolone + ifosfamide + |
| | carboplatin |
| ESHAP | etoposide + methylprednisolone + cytarabine + |
| | cisplatin |
| ICE | idarubicin + cytarabine + etoposide |
| IVE | ifosfamide + etoposide + epirubicin |
| MIME | mitoguazone + ifosfamide + methotrexate + |
| | etoposide |
| MINE | ifosfamide + mitoxantrone + etoposide |
| POMMB/ACE | vincristine + methotrexate + folinic acid + |
| | bleomycin + cisplatin then dactinomycin + |
| | cyclophosphamide + etoposide |
| R-ICE | rituximab + ifosfamide + carboplatin + etoposide |
| VIP | ifosfamide + etoposide + cisplatin |
| (V)-ICE | carboplatin + ifosfamide + etoposide + vincristine |
| PACEBOM | prednisolone + doxorubicin + cyclophosphamide + |
| | etoposide + bleomycin + vincristine + methotrexate |

Irinotecan (Campto ®, Camptosar ®)

| | |
|---|---|
| FOLFIRI | 5-FU + leucovorin + irinotecan |

Doxorubicin (Doxil ®, Adriamycin ®, Rubix ®)

| | |
|---|---|
| ABVD | doxorubicin + Bleo + vinblastine + dacarbazine |
| AC | doxorubicin + cyclophosphamide |
| A-CMF | doxorubicin followed by cyclophosphamide + |
| | methotrexate + 5-FU |
| AD | doxorubicin + dacarbazine |
| AIM | Doxorubicin + Ifosfamide + Mesna |
| AP | doxorubicin + cisplatin |
| ASHAP | doxorubicin + cisplatin + cytarabine + |
| | methylprednisolone |
| A-T-C | doxorubicin followed by paclitaxel followed by |
| | cyclophosphamide |
| BEACOPP | bleomycin + etoposide + doxorubicin + |
| | cyclophosphamide + vincristine + procarbazine + |
| | prednisone |
| CAV | doxorubicin + cyclophosphamide + vincristine |
| CADO | cyclophosphamide + vincristine + doxorubicin |
| CDEC | cisplatin + doxorubicin + etoposide + |
| | cyclophosphamide |
| ChlVPP/EVA | chlorambucil + vincristine + procarbazine + |
| hybrid | etoposide + prednisolone + doxorubicin + |
| | vinblastine |
| CHOP | cyclophosphamide + doxorubicin + vincristine + |
| | prednisone |
| CIDE | cisplatin + ifosfamide + doxorubicin + etoposide |
| COPP-ABVD | cyclophosphamide + vincristine + procarbazine + |
| alternating | prednisone + doxorubicin + bleomycin + |
| | vinblastine + dacarbazine |
| DVD | liposomal doxorubicin + vincristine + dexamethasone |
| ET-2 | ifosfamide + vincristine + doxorubicin + |
| | dactinomycin + cyclophosphamide |
| EVAIA | etoposide + vincristine + doxorubicin + ifosfamide + |
| | dactinomycin |
| FAC | 5-FU + doxorubicin + cyclophosphamide |
| FAM | 5-FU + doxorubicin + mitomycin |
| FAMTX | methotrexate + 5-FU + leucovorin + doxorubicin |
| MAID | Mesna + Doxorubicin + Ifosfamide + Dacarbazine |
| MOPP/ABV | Nitrogen mustard + vincristine + procarbazine + |
| hybrid | prednisone + doxorubicin + bleomycin + vinblastine |
| MVAC | methotrexate + vinblastine + doxorubicin + cisplatin |
| PIAF | cisplatin + doxorubicin + 5-FU + interferon alpha |
| Stanford V | doxorubicin + vinblastine + nitrogen mustard + |
| | vincristine + bleomycin + etoposide + prednisone |
| TAC | docetaxel + doxorubicin + cyclophosphamide |
| VACA | vincristine + doxorubicin + cyclophosphamide + |
| | dactinomycin |
| VAD | vincristine + doxorubicin + dexamethasone |
| VAIA | vincristine + doxorubicin + ifosfamide + dactinomycin |

Gemcitabine (Gemzar ®)

| | |
|---|---|
| GIN | gemcitabine + ifosfamide + vinorelbine |

TABLE 9

| | | nucleo- | nucleo- | | | | Patient/ | | |
|---|---|---|---|---|---|---|---|---|---|
| | Intron/ | tide | tide | codon | codon | Conse- | cell | | |
| Mutation | Exon | change | number | change | number | quence | line | Ascertainment | Reference |
| 1A>T | 4 | A>T | 1 | M>L | 1 | no protein | 7 | MCL | Camacho et |
| | | | | | | | | | al. 2002 |
| 67C>T | 4 | C>T | 67 | R>X | 23 | T | 8 | MCL | Camacho et |
| | | | | | | | | | al. 2002 |
| 146C>G | 5 | C>G | 146 | S>C | 49 | | 389 | Breast Cancer | Izatt et al. |
| | | | | | | | | | 1999 |
| 487C>T | 7 | C>T | 487 | Q>X | 163 | exon 7 | MCL-J | MCL | Schaffner et |
| | | | | | | skipped | | | al. 2000 |
| 544G>C | 8 | G>C | 544 | V>L | 182 | | 333 | Breast Cancer | Izatt et al. |
| | | | | | | | | | 1999 |
| 995A>G | 10 | A>G | 995 | Y>C | 332 | | B-CLL | B-CLL | Bullrich et al. |
| | | | | | | | | | 1999 |
| 1048G>A | 10 | G>A | 1048 | A>T | 350 | no protein | B-CLL2 | B-CLL | Stankovic et |
| | | | | | | | | | al. 1999 |
| 1055T>C | 10 | T>C | 1055 | I>T | 352 | no protein | B-CLL2 | B-CLL | Stankovic et |
| | | | | | | | | | al. 1999 |
| 1058delGT | 10 | delGT | 1058 | C>X | 353 | FS, T | B-CLL1 | B-CLL | Stankovic et |
| | | | | | | | | | al. 1999 |
| IVS10−6T>G | IVS10 | T>G | IVS10−6 | V>X | 419 | skip 11 | ATMb57 | Breast Cancer | Broeks et al. |
| | | | | | | | | | 2000 |
| IVS10−6T>G | IVS10 | T>G | IVS10−6 | V>X | 419 | skip 11 | ATMc214 | Breast Cancer | Broeks et al. |
| | | | | | | | | | 2000 |
| IVS10−6T>G | IVS10 | T>G | IVS10−6 | V>X | 419 | skip 11 | ATMc232 | Breast Cancer | Broeks et al. |
| | | | | | | | | | 2000 |
| IVS10−6T>G | IVS10 | T>G | IVS10−6 | V>X | 419 | skip 11 | na | Breast Cancer | Dork et al. |
| | | | | | | | | | 2001 |
| IVS10−6T>G | IVS10 | T>G | IVS10−6 | V>X | 419 | skip 11 | na | Breast Cancer | Dork et al. |
| | | | | | | | | | 2001 |
| 1300C>T | 12 | C>T | 1300 | P>S | 434 | | B-CLL38 | B-CLL | Stankovic et |
| | | | | | | | | | al. 2002 |
| 1563delAG | 12 | delAG | 1563 | R>X | 521 | FS, T | ATMc211 | Breast Cancer | Broeks et al. |
| | | | | | | | | | 2000 |
| 1563delAG | 12 | delAG | 1563 | R>X | 521 | FS, T | 13 | MCL | Camacho et |
| | | | | | | | | | al. 2002 |
| 1648A>G | 13 | A>G | 1648 | I>V | 550 | | na | Breast Cancer | Dork et al. |
| | | | | | | | | | 2001 |
| 1810C>T | 14 | C>T | 1810 | P>S | 604 | | na | Breast Cancer | Dork et al. |
| | | | | | | | | | 2001 |
| IVS14+2T>G | IVS14 | T>G | IVS14+2 | del33 | 601 | skip 15 | ATMb58 | Breast Cancer | Broeks et al. |
| | | | | | | | | | 2000 |
| 2114ACTCAT > | 15 | ACTCAT > | 2114 | YSS>FIP | 705-707 | | 482- | | Vorechovsky |
| TCATAC | | TCATAC | | | | | 89DFBC42 | | et al. 1996 |
| 2114insA | 15 | insA | 2114 | Y>X | 705 | FS, T | B-CLL34 | B-CLL | Stankovic et |
| | | | | | | | | | al. 2002 |
| 2119T>C | 15 | C>T | 2119 | S>P | 707 | | 379 | Breast Cancer | Izatt et al. |
| | | | | | | | | | 1999, |
| | | | | | | | | | Teraoka et al. |
| | | | | | | | | | 2002, Dork et |
| | | | | | | | | | al. 2001, |
| | | | | | | | | | Atencio et al. |
| | | | | | | | | | 2001 |
| 2119del4 | 15 | del4 | 2119 | S>X | 707 | FS, T | TPLL-5b3 | T-PLL | Vorechovsky |
| | | | | | | | | | et al. 1997 |
| 2250C>G | 16 | C>G | 2250 | N>K | 750 | | MCL-D | MCL | Schaffner et |
| | | | | | | | | | al. 2000 |
| 2362A>C | 17 | A>C | 2362 | S>R | 788 | | 49 | Breast Cancer | Atencio et al. |
| | | | | | | | | | 2001 |
| 2572T>C | 19 | T>C | 2572 | F>L | 858 | | 89 | Breast Cancer | Izatt et al. |
| | | | | | | | | | 1999 Dork et |
| | | | | | | | | | al. 2001 |
| | | | | | | | | | Rodriguez et |
| | | | | | | | | | al. 2002 |
| 2614C>T | 19 | C>T | 2614 | P>S | 872 | | na | Breast Cancer | Rodriguez et |
| | | | | | | | | | al. 2002 |
| 3118A>G | 23 | A>G | 3118 | M>V | 1040 | | NHL20 | NHL | Vorechovsky |
| | | | | | | | | | et al. 1997 |
| 3161C>G | 24 | C>G | 3161 | P>R | 1054 | 65% protein | B-CLL6 | B-CLL | Stankovic et |
| | | | | | | | | | al. 1999 |
| 3161C>G | 24 | C>G | 3161 | P>R | 1054 | | na | Breast Cancer | Stankovic et |
| | | | | | | | | | al. 1999 Dork |
| | | | | | | | | | et al. 2001 |
| 3246insG | 24 | insG | 3246 | H>X | 1083 | FS, T | AI18-FBC11 | | Vorechovsky |
| | | | | | | | | | et al. 1996 |
| 3556G>T | 26 | G>T | 3556 | E>X | 1186 | T | B-CLL40 | B-CLL | Stankovic et |
| | | | | | | | | | al. 2002 |
| 3802delG | 28 | delGT | 3802 | V>X | 1268 | FS, T | na | Breast Cancer | Dork et al. |
| | | | | | | | | | 2001 |
| 3873del120 | 28 | del120 | 3873 | del40 | 1292 | | T-PLL21 | T-PLL | Stilgenbauer |
| | | | | | | | | | et al. 1997 |
| 3910del7 | 28 | del7 | 3910 | R>X | 1304 | FS, T | B-CLL5 | B-CLL | Stankovic et |
| | | | | | | | | | al. 1999 |
| 3993ins29 | 29 | ins29 | 3993 | I>X | 1332 | | Bat | T-PLL | Stoppa- |
| | | | | | | | | | Lyonnet et al. |
| | | | | | | | | | 1998 |
| 3994ins190 | 29 | ins190 | 3994 | I>X | 1332 | FS, T | B-CLL6 | B-CLL | Stankovic et |
| | | | | | | | | | al. 1999 |
| 4081C>T | 29 | C>T | 4081 | Q>X | 1361 | T | MCL-G | MCL | Schaffner et |
| | | | | | | | | | al. 2000 |
| 4138C>T | 30 | C>T | 4138 | H>Y | 1380 | | na | Breast Cancer | Teraoka et al. |
| | | | | | | | | | 2002 |
| 4138C>T | 30 | C>T | 4138 | H>Y | 1380 | | 45 | Breast Cancer | Atencio et al. |
| | | | | | | | | | 2001 |
| 4148C>T | 30 | C>T | 4148 | S>L | 1383 | | na | Breast Cancer | Teraoka et al. |
| | | | | | | | | | 2002 |
| 4174insC | 30 | insC | 4174 | Y>X | 1392 | FS, T | TPLL-1c8 | T-PLL | Vorechovsky |
| | | | | | | | | | et al. 1997 |
| 4182ins29 | 30 | ins29 | 4182 | N>X | 1395 | FS, T | 1 | MCL | Camacho et |
| | | | | | | | | | al. 2002 |
| 4220T>C | 30 | T>C | 4220 | I>T | 1407 | | TPLL-5b3 | T-PLL | Vorechovsky |
| | | | | | | | | | et al. 1997 |
| 4246C>T | 31 | C>T | 4246 | Q>X | 1416 | T | N114 | Breast Cancer | FitzGerald et |
| | | | | | | | | | al. 1997 |
| 4258C>T | 31 | C>T | 4258 | L>F | 1420 | | 66 | Breast Cancer | Izatt et al. |
| | | | | | | | | | 1999, Dork et |
| | | | | | | | | | al. 2001 |
| 4387T>C | 31 | T>C | 4387 | F>S | 1463 | | NHL34 | NHL | Vorechovsky |
| | | | | | | | | | et al. 1997 |
| 4393insA | 31 | insA | 4393 | L>X | 1465 | FS, T | B-CLL34 | B-CLL | Stankovic et |
| | | | | | | | | | al. 2002 |
| 4400A>G | 31 | A>G | 4400 | D>G | 1467 | | 45 | Breast Cancer | Atencio et al. |
| | | | | | | | | | 2001 |
| 4709T>C | 33 | T>C | 4709 | V>A | 1570 | | 618 | Breast Cancer | Izatt et al. |
| | | | | | | | | | 1999 |
| 4709T>C | 33 | T>C | 4709 | V>A | 1570 | | na | Breast Cancer | Dork et al. |
| | | | | | | | | | 2001 |
| 4736del2 | 33 | del2 | 4736 | Q>X | 1579 | FS, T | N119 | Breast Cancer | FitzGerald et |
| | | | | | | | | | al. 1997 |
| 4829delG | 34 | delG | 4829 | R>X | 1610 | FS, T | B-CLL41 | B-CLL | Stankovic et |
| | | | | | | | | | al. 2002 |
| 5044G>C | 36 | G>C | 5044 | D>H | 1682 | | TPLL-1b8 | T-PLL | Vorechovsky |
| | | | | | | | | | et al. 1997 |
| 5071A>C | 36 | A>C | 5071 | S>R | 1691 | | B-CLL | B-CLL | Bullrich et al. |
| | | | | | | | | | 1999 |
| 5071A>C | 36 | A>C | 5071 | S>R | 1691 | | na | Breast Cancer | Dork et al. |
| | | | | | | | | | 2001 |
| 5071A>C | 36 | A>C | 5071 | S>R | 1691 | | na | Breast Cancer | Dork et al. |
| | | | | | | | | | 2001 |
| 5071A>C | 36 | A>C | 5071 | S>R | 1691 | | na | Breast Cancer | Teraoka et al. |
| | | | | | | | | | 2002 |
| IVS36+44T>C | IVS36 | T>C | IVS36+44 | na | na | | T-PLL8 | T-PLL | Yuille et al. |
| | | | | | | | | | 1998 |
| 5309C>G | 37 | C>G | 5309 | S>X | 1770 | T | T-PLL11 | T-PLL | Stilgenbauer |
| | | | | | | | | | et al. 1997 |
| 5464G>A | 38 | G>A | 5464 | E>Q | 1822 | absent | B-CLL1 | B-CLL | Stankovic et |
| | | | | | | protein | | | al. 2002 |
| 5558A>T | 39 | A>T | 5558 | D>V | 1853 | | na | Breast Cancer | Dork et al. |
| | | | | | | | | | 2001 |
| 5729T>A | 40 | T>A | 5729 | L>H | 1910 | | TPLL1b2 | T-PLL | Vorechovsky |
| | | | | | | | | | etal. 1997 |
| IVS40−22del31 | IVS40 | del31 | IVS40−22 | R>X | 1921 | FS, T | T-PLL5 | T-PLL | Yuille et al. |
| | | | | | | | | | 1998 |
| 5858C>G | 41 | C>G | 5858 | T>R | 1953 | | B-CLL-C | B-CLL | Schaffner et |
| | | | | | | | | | al. 1999 |
| 6055T>G | 43 | T>G | 6055 | Y>D | 2019 | | B-CLL37 | B-CLL | Stankovic et |
| | | | | | | | | | al. 2002 |
| 6067G>A | 43 | G>A | 6067 | G>R | 2023 | | na | Breast Cancer | Teraoka et al. |
| | | | | | | | | | 2002 |
| 6100C>T | 44 | C>T | 6100 | R>X | 2034 | T | na | Breast Cancer | Teraoka et al. |
| | | | | | | | | | 2002 |
| 6116A>G | 44 | A>G | 6116 | E>G | 2039 | | T-PLL6 | T-PLL | Yuille et al. |
| | | | | | | | | | 1998 |
| 6278delC | 45 | delC | 6278 | P>X | 2093 | FS, T | B-CLL4 | B-CLL | Stankovic et |
| | | | | | | | | | al. 2002 |
| 6295A>C | 45 | A>C | 6295 | H>Y | 2099 | | B-CLL39 | B-CLL | Stankovic et |
| | | | | | | | | | al. 2002 |
| IVS46+1G>A | IVS46 | G>A | IVS46+1 | del35 | 2116 | | T-PLL, Dia | T-PLL | Stoppa- |
| | | | | | | | | | Lyonnet et al. |
| | | | | | | | | | 1998 |
| 6490G>A | 47 | G>A | 6490 | E>K | 2164 | | TPLL1d4 | T-PLL | Vorechovsky |
| | | | | | | | | | et al. 1997 |
| 6638delA | 48 | delA | 6638 | K>X | 2213 | FS, T | MCL-H | MCL | Schaffner et |
| | | | | | | | | | al. 2000 |
| 6709delAA | 48 | delAA | 6709 | K>X | 2237 | | MCL-E | MCL | Schaffner et |
| | | | | | | | | | al. 2000 |
| 6820G>A | 49 | G>A | 6820 | A>K | 2274 | 65% protein | B-CLL4 | B-CLL | Stankovic et |
| | | | | | | | | | al. 1999 |
| 6820G>A | 49 | G>A | 6820 | A>K | 2274 | | na | Breast Cancer | Dork et al. |
| | | | | | | | | | 2001 |
| 6860G>C | 49 | G>C | 6860 | G>A | 2287 | | na | Breast Cancer | Dork et al. |
| | | | | | | | | | 2001 |
| 7187C>G | 51 | C>G | 7187 | T>M | 2396 | | TPLL-1c8 | T-PLL | Vorechovsky |
| | | | | | | | | | et al. 1997 |
| 7258G>C | 51 | G>C | 7258 | A>P | 2420 | | B-CLL-G | B-CLL | Schaffner et |
| | | | | | | | | | al. 1999 |
| 7268A>G | 51 | A>G | 7268 | E>G | 2419 | | MCL-B | MCL | Schaffner et |
| | | | | | | | | | al. 1999 |
| 7271T>G | 51 | T>G | 7271 | V>G | 2424 | | TPLL1c10 | T-PLL | Vorechovsky |
| | | | | | | | | | et al. 1997 |
| 7271T>G | 51 | T>G | 7271 | V>G | 2424 | | B-CLL | B-CLL | Bullrich et al. |
| | | | | | | | | | 1999 |
| 7315G>A | 52 | G>A | 7315 | A>T | 2451 | | B-CLL7 | B-CLL | Stankovic et |
| | | | | | | | | | al. 2002 |
| 7325A>C | 52 | A>C | 7325 | Q>P | 2442 | | TPLL-1a9 | T-PLL | Vorechovsky |
| | | | | | | | | | et al. 1997 |
| 7349insT | 52 | insT | 7349 | L>X | 2450 | | MCL-F | MCL | Schaffner et |
| | | | | | | | | | al. 2000 |
| 7390T>C | 52 | T>C | 7390 | C>R | 2464 | | na | Breast Cancer | Dork et al. |
| | | | | | | | | | 2001 |
| 7390T>C | 52 | T>C | 7390 | C>R | 2464 | | na | Breast Cancer | Dork et al. |
| | | | | | | | | | 2001 |
| 7253insGAA | 51 | insGAA | 7253 | insK | 2418 | | MCL-B | MCL | Schaffner et |
| | | | | | | | | | al. 2000 |
| 7456C>G | 52 | C>G | 7456 | R>G | 2486 | | Bul | T-PLL | Stoppa- |
| | | | | | | | | | Lyonnet et al. |
| | | | | | | | | | 1998 |
| 7511del62 | 52 | del62 | 7511 | M>X | 2504 | FS, T | B-CLL42 | B-CLL | Stankovic et |
| | | | | | | | | | al. 2002 |
| 7636del9 | 54 | del9 | 7636 | del3 | 2547 | | TPLL-1d5 | T-PLL | Vorechovsky |
| | | | | | | | | | et al. 1997 |
| 7636del9 | 54 | del9 | 7636 | del3 | 2547 | | AL7-FBC33 | | Vorechovsky |
| | | | | | | | | | et al. 1996 |
| 7775C>G | 54 | C>G | 7775 | S>C | 2592 | | na | Breast Cancer | Dork et al. |
| | | | | | | | | | 2001 |
| IVS54+8G>T | IVS54 | G>T | IVS54+8 | na | na | | na | Breast Cancer | Teraoka et al. |
| | | | | | | | | | 2002 |
| 7865C>T | 55 | C>T | 7865 | A>V | 2622 | splicing | B-CLL | B-CLL | Bullrich et al. |
| | | | | | | | | | 1999 |
| 7880insT | 55 | insT | 7880 | Y>X | 2627 | FS, T | TPLL-6c4 | T-PLL | Vorechovsky |
| | | | | | | | | | et al. 1997 |
| 7890insTATTA | 55 | insTATTA | 7890 | A>X | 2631 | FS, T | MCL-J | MCL | Schaffner et |
| | | | | | | | | | al. 2000 |
| 8084G>C | 57 | G>C | 8084 | G>A | 2695 | | TPLL-1a8 | T-PLL | Vorechovsky |
| | | | | | | | | | et al. 1997 |
| 8084G>C | 57 | G>C | 8084 | G>A | 2695 | | B-CLL7 | B-CLL | Stankovic et |
| | | | | | | | | | al. 1999 |
| 8150A>T | 57 | A>T | 8150 | K>M | 2717 | low protein | 12 | MCL | Camacho et |
| | | | | | | | | | al. 2002 |
| 8165T>G | 58 | T>G | 8165 | L>R | 2722 | | TPLL-1b4 | T-PLL | Vorechovsky |
| | | | | | | | | | et al. 1997 |
| 8174A>T | 58 | A>T | 8174 | D>V | 2725 | | TPLL-1b7 | T-PLL | Vorechovsky |
| | | | | | | | | | et al. 1997 |
| 8174A>T | 58 | A>T | 8174 | D>V | 2725 | | 6 | MCL | Camacho et |
| | | | | | | | | | al. 2002 |
| 8174A>G | 58 | A>G | 8174 | D>G | 2725 | | T-PLL13 | T-PLL | Stilgenbauer |
| | | | | | | | | | et al. 1997 |
| 8194T>C | 58 | T>C | 8194 | F>L | 2732 | | TPLL-5a6 | T-PLL | Vorechovsky |
| | | | | | | | | | et al. 1997 |
| 8266A>T | 58 | A>T | 8266 | K>X | 2451 | T | B-CLL35 | B-CLL | Stankovic et |
| | | | | | | | | | al. 2002 |
| 8293G>A | 59 | G>A | 8293 | G>S | 2765 | | 31 | Breast Cancer | Izatt et al. |
| | | | | | | | | | 1999 |
| 8314G>A | 59 | G>A | 8314 | G>R | 2772 | | na | Breast Cancer | Dork et al. |
| | | | | | | | | | 2001 |
| 8413delA | 59 | delA | 8413 | M>X | 2805 | FS, T | B-CLL-A | B-CLL | Schaffner et |
| | | | | | | | | | al. 1999 |
| IVS59+1G>T | IVS59 | G>T | IVS59+1 | na | 2757 | 59 skipped | MCL-A | MCL | Schaffner et |
| | | | | | | | | | al. 1999 |
| 8430del3 | 60 | del3 | 8430 | del1 | 2810 | | TPLL-t1a5 | T-PLL | Vorechovsky |
| | | | | | | | | | et al. 1997 |
| 8473C>T | 60 | C>T | 8473 | Q>X | 2825 | T | BRCA51 | Breast Cancer | FitzGerald et |
| | | | | | | | | | al. 1997 |
| 8494C>T | 60 | C>T | 8494 | R>C | 2832 | | Granta519 | | Vorechovsky |
| | | | | | | | | | et al. 1997 |
| 8534GG>AA | 60 | GG>AA | 8534 | W>X | 2845 | T | HT144 | melanoma | Ramsay et al. |
| | | | | | | | | | 1998 |
| 8535G>A | 60 | G>A | 8535 | W>X | 2845 | T | BRCA258 | Breast Cancer | FitzGerald et |
| | | | | | | | | | al. 1997 |
| 8613del3 | 61 | del3 | 8613 | R>S, delH | 2871 | | TPLL-1a1 | T-PLL | Vorechovsky |
| | | | | | | | | | et al. 1997 |
| 8668C>G | 61 | C>G | 8668 | L>V | 2890 | | TPLL-6b1 | T-PLL | Vorechovsky |
| | | | | | | | | | et al. 1997 |
| 8668C>G | 61 | C>G | 8668 | L>V | 2890 | | T-PLL3 | T-PLL | Yuille et al. |
| | | | | | | | | | 1998 |
| 8734A>G | 62 | A>G | 8734 | R>G | 2912 | | na | Breast Cancer | Teraoka et al. |
| | | | | | | | | | 2002 |
| 8839A>C | 63 | A>C | 8839 | T>S | 2947 | | B-CLL36 | B-CLL | Stankovic et |
| | | | | | | | | | al. 2002 |
| 8968G>T | 64 | G>T | 8968 | E>X | 2990 | T | Br60 | Breast Cancer | Chen et al. |
| | | | | | | | | | 1998 |
| 9016G>C | 65 | G>C | 9016 | A>P | 3006 | | T-PLL15 | T-PLL | Stilgenbauer |
| | | | | | | | | | et al. 1997 |
| 9022C>T | 65 | C>T | 9022 | R>C | 3008 | | T-PLL3 | T-PLL | Stilgenbauer |
| | | | | | | | | | et al. 1997 |
| 9022C>T | 65 | C>T | 9022 | R>C | 3008 | | T-PLL4 | T-PLL | Yuille et al. |
| | | | | | | | | | 1998 |
| 9022C>T | 65 | C>T | 9022 | R>C | 3008 | | MCL-C | MCL | Schaffner et |
| | | | | | | | | | al. 2000 |
| 9023G>A | 65 | G>A | 9023 | R>H | 3008 | | B-CLL-D | B-CLL | Schaffner et |
| | | | | | | | | | al. 1999 |
| 9023G>A | 65 | G>A | 9023 | R>H | 3008 | reduced | 2 | MCL | Camacho et |
| | | | | | | protein | | | al. 2002 |
| 9031A>G | 65 | A>G | 9031 | M>V | 3011 | | na | Breast Cancer | Teraoka et al. |
| | | | | | | | | | 2002 |
| 9054A>C | 65 | A>C | 9054 | K>D | 3018 | | B-CLL-E | B-CLL | Schaffner et |
| | | | | | | | | | al. 1999 |
| 9139C>T | 65 | C>T | 9139 | R>X | 3047 | T | TPLL-BJ01 | T-PLL | Vorechovsky |
| | | | | | | | | | et al. 1997 |
| 9139C>T | 65 | C>T | 9139 | R>X | 3047 | T | B-CLL-B | B-CLL | Schaffner et |
| | | | | | | | | | al. 1999 |

### REFERENCES

Camacho et al. 2002 Blood. 2002 Jan. 1; 99(1):238-44.
Izatt et al. 1999 Genes Chromosomes Cancer. 1999 December; 26(4):286-94.
Schaffner et al. 2000 Proc Natl Acad Sci USA. 2000 Mar. 14; 97(6):2773-8
Bullrich et al. 1999 Cancer Res Jan. 1; 59(1):24-7.
Stankovic et al. 1999 Lancet. 1999 Jan. 2; 353(9146):26-9
Broeks et al. 2000 Am J Hum Genet. 2000 February; 66(2):494-500.
Dork et al. 2001 Cancer Res October 15; 61(20):7608-15.
Stankovic et al. 2002 Leuk Lymphoma. 2002 August; 43(8):1563-71
Vorechovsky et al. 1996 Cancer Res. 1996 Sep. 15; 56(18):4130-3
Atencio et al. 2001 Environ Mol Mutagen. 2001; 38(2-3):200-8
Vorechovsky et al. 1997 Nat Genet. 1997 September; 17(1):96-9
Rodriguez et al. 2002 Genes Chromosomes Cancer 2002 February; 33(2):141-9
Stilgenbauer et al. 1997 Nat Med 1997 October; 3(10):1155-9
Stoppa-Lyonnet et al. 1998 Blood 1998 May 15; 91(10):3920-6.
FitzGerald et al. Nat Genet 1997 March; 15(3):307-10.
Yuille et al. 1998 Oncogene 1998 Feb. 12; 16(6):789-96.
Schaffner et al. 1999 Blood. 1999 Jul. 15; 94(2):748-53
Teraoka et al. Am J Hum Genet. 1999 June; 64(6):1617-31.
Teraoka et al. Cancer. 2001 Aug. 1; 92(3):479-87
Ramsay et al. 1998 Radiother Oncol. 1998 May; 47(2):125-8
Chen et al. 1998 Cancer Res 1998 Apr. 1; 58(7):1376-9

1-13. (canceled)

14. A method of treatment of cancer in an individual comprising;
administering a DNA damaging cancer therapy and a DNA-PKcs inhibitor to said individual,
wherein said cancer has an ATM deficient phenotype.

15. A method of determining the susceptibility of a cancer condition in an individual to cancer therapy, said method comprising
identifying a cancer cell obtained from the individual as having an ATM deficient phenotype,
wherein said cancer therapy comprises a combination of a DNA-PKcs inhibitor and a DNA damaging cancer therapy and
wherein the identification of the cancer cell obtained from the individual as a cancer cell having an ATM deficient phenotype is indicative of the cancer being susceptible to said cancer therapy.

16. A method according to claim 14 or 15 wherein the DNA damaging cancer therapy induces DNA double strand breaks in cellular DNA.

17. A method according to claim 14 or claim 15 to 16 wherein the DNA damaging cancer therapy is irradiation therapy.

18. A method according to claim 14 or claim 15 to 16 wherein the DNA damaging cancer therapy is one or more DNA damaging chemotherapeutic agents.

19. A method according to claim 18 wherein the one or more DNA damaging chemotherapeutic agents are selected from the group consisting of bleomycin, doxorubicin, etoposide, irinotecan, topotecan, rubitecan, gemcitabine, temozolomide, DTIC (dacarbazine), cisplatin, oxaliplatin and carboplatin and yondelis.

20. A method according to claim 18 wherein the DNA damaging chemotherapeutic therapy is a combination of DNA damaging chemotherapeutic agents shown in Table 8.

21. A method according to claim 14 or claim 15 wherein the DNA-PKcs inhibitor is an aryl-morpholino compound, a benzochromenone, a morpholino-salicylaldehyde, or a morpholino-benzophenone.

22. A method according to claim 21 wherein the DNA-PKcs inhibitor is selected from the group consisting of 1-(2-hydroxy-4-morpholin-4-yl-phenyl)ethanone, 2-amino-N-[4-(2-morpholin-4-yl-4-oxo-4H-pyrido[1,2-a]pyrimidin-9-yl)-dibenzothiophen-1-yl]-acetamide, 9-dibenzothiophen-4-yl-2-morpholin-4-yl-pyrido[1,2-a]pyrimidin-4-one, 2-amino-N-[4-(2-morpholin-4-yl-4-oxo-1,4-dihydro-quinolin-8-yl)-dibenzothiophen-1-yl]-acetamide, 8-dibenzothiophen-4-yl-2-morpholin-4-yl-1H-quinolin-4-one, 3-amino-N-[4-(2-morpholin-4-yl-4-oxo-4H-pyrido[1,2-a]pyrimidin-9-yl)-dibenzothiophen-1-yl]-propionamide, 3-amino-N-[4-(2-morpholin-4-yl-4-oxo-1,4-dihydro-quinolin-8-yl)-dibenzothiophen-1-yl]-propionamide, 2-(morpholin-4-yl)-benzo[h]chromen-4-one, 8-Dibenzothiophen-4-yl-2-morpholin-4-yl-chromen-4-one, 2-amino-chromen-4-one, 2-hydroxy-4-morpholin-4-yl-benzaldehyde, and 1-(2-Hydroxy-4-morpholin-4-yl-phenyl)-phenyl-methanone.

23. A method according to claim 14 or claim 15 wherein the DNA-PKcs inhibitor has the formula (I): 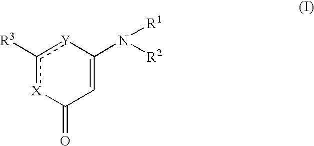
wherein:
R¹ and R² are independently hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
X and Y are selected from CR⁴ and O, O and CR′⁴ and NR″⁴ and N, where the unsaturation is in the appropriate place in the ring, and where one of R³ and R⁴ or R′⁴ is an optionally substituted C₃₋₂₀ heteroaryl or C₅₋₂₀ aryl group, and the other of R³ and R⁴ or R′⁴ is H, or R³ and R⁴ or R″⁴ together are -A-B-, which collectively represent a fused optionally substituted aromatic ring;
except that when X and Y are CR⁴ and O, R³ and R⁴ together form a fused benzene ring, and R¹ and R² together with the N to which they are attached form a morpholino group, then the fused benzene does not bear as a sole substituent a phenyl substituent at the 8-position.

24. A method according to claim 23 wherein the DNA-PKcs inhibitor has the formula (II): 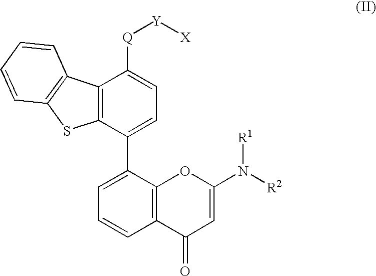
wherein:
R¹ and R² are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
Q is —NH—C(═O)— or —O—;
Y is an optionally substituted C₁₋₅ alkylene group;
X is selected from SR³ or NR⁴R⁵, wherein, R³, or R⁴ and R⁵ are independently selected from hydrogen, optionally substituted C₁₋₇ alkyl, C₅₋₂₀ aryl, or C₃₋₂₀ heterocyclyl groups, or R⁴ and R⁵ may together form, along with the nitrogen atom to which they are attached, an optionally substituted heterocyclic ring having from 4 to 8 ring atoms;
if Q is —O—, X is additionally selected from —C(═O)—NR⁶R⁷, wherein R⁶ and R⁷ are independently selected from hydrogen, optionally substituted C₁₋₇ alkyl, C₅₋₂₀ aryl, or C₃₋₂₀ heterocyclyl groups, or R⁶ and R⁷ may together form, along with the nitrogen atom to which they are attached, an optionally substituted heterocyclic ring having from 4 to 8 ring atoms; and
if Q is —NH—C(—O)—, —Y—X may additionally selected from C₁₋₇ alkyl.

25. A method according to claim 24 wherein the DNA-PKcs inhibitor is selected from the group consisting of 8-aryl-2-morpholin-4-yl-1-benzopyran-4-one and 2-(4-ethyl-piperazin-1-yl)-N-[4-(2-morpholin-4-yl-4-oxo-4H-1-benzopyran-8-yl)-dibenzothiophen-1-yl]-acetamide.

26. A method according to claim 23 wherein the DNA-PKcs inhibitor has the formula (III): 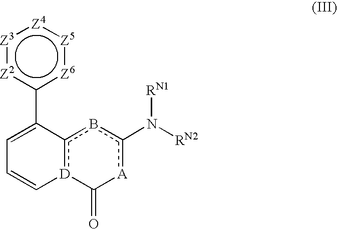
wherein:
A, B and D are respectively selected from the group consisting of:
(i) CH, NH, C;
(ii) CH, N,N; and
(iii) CH, O, C;
the dotted lines represent two double bonds in the appropriate locations;
R^{N1} and R^{N2} are independently selected from hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
Z², Z³, Z⁴, Z⁵ and Z⁶, together with the carbon atom to which they are bound, form an aromatic ring;
Z² is selected from the group consisting of CR², N, NH, S, and O; Z³ is CR³; Z⁴ is selected from the group consisting of CR⁴, N, NH, S, and O; Z⁵ is a direct bond, or is selected from the group consisting of O, N, NH, S, and CH; Z⁶ is selected from the group consisting of O, N, NH, S, and CH;
R² is H;
R³ is selected from halo or optionally substituted C₅₋₂₀ aryl;
R⁴ is selected from the group consisting of H, OH, NO₂, NH₂ and Q-Y—X, where Q is —NH—C(═O)— or —O—;
Y is an optionally substituted C₁₋₅ alkylene group;
X is selected from SR^{S1} or NR^{N3}R^{N4}, wherein,
R^{S1}, or R^{N3} and R^{N4} are independently selected from hydrogen, optionally substituted C₁₋₇ alkyl, C₅₋₂₀ aryl, or C₃₋₂₀ heterocyclyl groups, or R^{N3} and R^{N4} may together form, along with the nitrogen atom to which they are attached, an optionally substituted heterocyclic ring having from 4 to 8 ring atoms;
if Q is —O—, X may additionally be selected from —C(═O)—NR^{N5}R^{N6}, wherein R^{N5} and R^{N6} are independently selected from hydrogen, optionally substituted C₁₋₇ alkyl, C₅₋₂₀ aryl, or C₃₋₂₀ heterocyclyl groups, or R^{N5} and R^{N6} may together form, along with the nitrogen atom to which they are attached, an optionally substituted heterocyclic ring having from 4 to 8 ring atoms and
if Q is —NH—C(═O)—, —Y—X may be additionally selected from C₁₋₇ alkyl.

27. A method according to claim 26 wherein the DNA-PKcs inhibitor is selected from the group consisting of 8-aryl-2-morpholin-4-yl-1H-quinolin-4-one, 9-aryl-2-morpholin-4-yl-9H-pyrido[1,2-a]pyrimidin-4-one, 9-aryl-2-morpholin-4-yl-quinolizin-4-one and 5-aryl-3-morpholin-4-yl-2-benzopyran-1-one.
